SEQUENCE LISTING (173 sequences)

SEQ ID NO: 1 - DNA, 233 bp, Nicotiana tabacum
  plasmid a1-1-14
gtaacgaggtacagaagcattgtattacaatgttattcatcacatgatattaaagaggtagggtttantttgttggacattgaagctaaaatttctctttaacacttaatgaaatatttaatttagattcaggctcacagacttgacgctgctatttttttactcagtaagatcatctttatctgtagtctgtaccaataataaaagcccaaacccctttaaccacattcatc

SEQ ID NO: 2 - DNA, 314 bp, Nicotiana tabacum
  plasmid a10-2-12
aatggaagaggtcaagaagaatgtaaatggcttttcagagaccattgaggttttgagtcagcacgccaagaaatgtaaccaggatattaggatggcacgagcagtgatcttgcggaagattgtcagtcaacccagtagctngaatcagggcattggtatgttctttgattgacagtagntgtcttgnngattttctttttgtttatataccatgtatgtttgtaaaaagttggtccaattatgttctgttggatctgttgatttgagatttttgacccctgcagaaaattaagttatagtcctcattttgttag

SEQ ID NO: 3 - DNA, 286 bp, Nicotiana tabacum
  plasmid a10-4-1
ggcagcggctgcggaggatgtgggatctacccagacttggagaagtccatacctttaccatcgttgatggtgttgctcccatgaagagctttgaggaatttggagagaaagcagcagaaggaggaaatggctgcaaatgcggatcaaactgcacctgtgacccttgcaattgttaagataattctcttgtgattccacaataatgtgtgtgttttctgtaataataaggataaaactacagctagccatggaactgattgtcagtttttaggtttgtttgttctga

SEQ ID NO: 4 - DNA, 286 bp, Nicotiana tabacum
  plasmid a10-4-12
gacatcagctgttggagctcaagactttcctcctgtttcacaatatgactataaatanttgcaacttcagctgttggagctcaacatttgcaagtgattattggctttgaagagaacttaatttattggttgtgacttggtggcaaattatgtgttttcaagtagtaatttgccttgtgcctctatgttttcaantagtaatttgccttcgcgagttgattacatgagaaatcagattctcagtctttgtgtagtaattatttgggctggtgccatcagccaagtg

SEQ ID NO: 5 - DNA, 278 bp, Nicotiana tabacum
  plasmid a10-4-15
ctacaaaagaaaggttatttatacaatatgcattgtaaaaaatcaaccgttaatacaatgggcngcataacatataatataagattttgataacctaatgaccaacaacacttatttatataatatgtggaaaagatgcatccaactatcacagatataacatccaaaggctatacttaatttctnctaaataacaaacacacacttaatccgtcactcctcgtgtgtacaagcaatagtccccaatttagttgtcatcctctaacattcaatattcc

SEQ ID NO: 6 - DNA, 349 bp, Nicotiana tabacum
  plasmid a14-1-1 ; homology with a serine carboxypeptidase
gcagaaagattttgggggnggcaccatctggtttctttcacagtagatggtgaggagaaaggaattcaaaagagctatggacctctgactttcctcaaagtcccatgatgcaggtcatatggtgccaatggaccaaccaaaggcagcactcgaaatgctccagaggtggactgctcaaggcaaattgtcctaagaagattatcttgctcacatgtgaagcatcaatttaagaaccacacttaactgaaacagatttaacatttttccagctttaaaattccatcaaaacatagaaaatcatgtagatacatttcaccttttcaggttaccctgaaatctgtcaatgaaa

SEQ ID NO: 7 - DNA, 367 bp, Nicotiana tabacum
  plasmid a14-1-3
gtggaggaaaggttcagggaaggttgtcggagtcaatgccgccgatgccgggaaagtttacctgagaaatgaaactgccggaattttgacggttggcgacatgaggaacgttaagtcaccgttagagataacggagggtgacgacacgtggtgggacgcggacgccgttacaatcgaggagcagtttgacggttcaaataaaactagtcaaattgaacgagtttcactgactcggtgaatgaatgatctaaaaagggtaaaatcgtaaatgacaaaggcgaaatgtgaaggaacgaacactcgtccgtgtttgtctgtaaatataattattttcaataattattggaaatgataatttaatatttgg

SEQ ID NO: 8 - DNA, 389 bp, Nicotiana tabacum
  plasmid a14-1-4
ggaagaagaaagagaaagggctgagaaagagaaagagaaagagaaagaagcagctgctgaagaagccaagattactgataaagtgaacgaaaatgagaagtcggagagtaatattgtcaaggaaaatccagagggtaatggtgttaaggaaaatggtaagtcggaaaataatgttgtcaaggaaaatggtgatgttagtaaaggttgatcatgaaatgattgattaattaggagttccacttaaaactaggatccaataattttgaatagttttgctgtgttcacattgttgactttgttattcaaactattcggatggaagtagtggatgtcgcaaattacatttagtattactaccttcttgtgaaagtaacattttcataatttag

SEQ ID NO: 9 - DNA, 317 bp, Nicotiana tabacum
  plasmid a18-1-5 ; homology with EREBP-1
ggacatacnanacggcggaggatgcggcgttggcgtatgacaaggcggcgtatcgaattcggggatcgcgtgcagtgttgaatttcccgttgagggttaattcgggtgaaccggaaccggttcgggttggttcgaanaggtcgtcaatttcgccggagagttcttcctcgtcgtcgtcggaaaatatttcgacaaagaggacgaagaaggttgcccnnctatacagctgagggttaatttgggaatttcaaaattgttcaattccatgaacaggttgagttcaatattttatttcatttcctctcctcntagaaatt

SEQ ID NO: 10 - DNA, 276 bp, Nicotiana tabacum
  plasmid a18-1-8
ctgagctaggagagcacacagggccttagttcaagtggaaaaggtggaaggacttgtgattaagtcacgggtttgagctacgtgccatgcgaattaagcttggtatttaagtggagtagggtagaggggtggacccattatccgagtttcgaatgctgcagttgtncctagacagatttctcggtcctcaaaataaaataaaataaatgagcttggagaataaactccatttttgtgacagtacaatcttctgcataaacatanctcaaaaagtgt

SEQ ID NO: 11 - DNA, 293 bp, Nicotiana tabacum
  plasmid a18-3-2
gatgtacctgaagccactgctatggantatgttggaggtattttatcaacaattggcnaaanatgtatgctcgattttgtattttgattcntaaanttgataannnngagntgaantcgactgtattttgcaagngtagttatatctttaatcttgtttcataaaatgcatgtgtgattgttattttagtcgatagaaaaaagaaagacccngtatagtttgttgatctgtgctgcagtttttgacagccaatgctgttttttaggttacaatatgnagttgattttctattg

SEQ ID NO: 12 - DNA, 290 bp, Nicotiana tabacum
  plasmid a18-3-3 ; homology with EIF-5A (initiation factor 5A2)
ttaaaggtggatttgaggaaggaaaggatcttgtgttgtctgtgatgtctgcaatgggtgaagagcagattgccgctgttaaggacattggtaccaagaactagtcgcgcattctgcagcataaataatttgctttagccaagacattttatatcttaatcgtggtactttgatatccgttgattatgaactcgacttatatcctattggcatggcttgaatagttgaactttatggtttgtctggtaagacagaactggatttgatagcagaagtgatttatatgaatg

SEQ ID NO: 13 - DNA, 260 bp, Nicotiana tabacum
  plasmid a18-4-6
tgatgatggctgggttcactccatccccaattagtngnaacgtntatgantngatccagaattttatcaagcnatatagtgnaaggnacaaagccaaggggggggcaggtgcaatncattttgggtggggaganaagagnntgattgttgcttnagcttgggaatagttacnaagtatggttttctcatataaacccacaatgtgcatcgaatcaacttgtattgacatctgactttgtgataatattcagtgtttatga

SEQ ID NO: 14 - DNA, 269 bp, Nicotiana tabacum
  plasmid a19-3-1
cgtgatagttttttcgcgacttgattagaagcaaatcagcaatagataagggacttgtataaaagataggtagcaaaatatactgtcctcttcgtcctctgccttttttttctttttaactttgattttacagccatctctggtaaaagttctgatttctctgggctcagttttgttaatcaatataaatcaatataaaaacagcttgcttttctatgtttnggttgatttagatatgcaaatncttggtagagctgtttctctttncc

SEQ ID NO: 15 - DNA, 268 bp, Nicotiana tabacum
  plasmid a19-3-3
gagtaaaattcatatttgataattatacaaggaaattacattcttaaagaagtgattttgatttgagttccaagatttggtgaagttactaaacagattttgagttcctaacttgtgcgcaatgctggataactcagccattttaatattctagtactccattaatttattgtttcttaacctatgtgtatgtttttcctgccgcagcaactttagttgatttcagagtattcgttttgatttgctcgaaaattgaaaaggacttgcc

SEQ ID NO: 16 - DNA, 269 bp, Nicotiana tabacum
  plasmid a19-3-4
caaggcagagagacttgaataaaggggatcatgaggattgaaccttacacggtaagatgtaaaataacagtnctatcacggaattactattcaatcctcaaaatgataagttgtncaaataaatggggattataagatnccttttatctttgcggaagggggtgattttgtatnctngggatgtgtaactgttgaataaaattgtgtgaaatccattgttcataatgtacgaaatttcaaaactattatatatgcgggactttaattta

SEQ ID NO: 17 - DNA, 265 bp, Nicotiana tabacum
  plasmid a19-3-9
aataaactatgaagtcgagatatgaatcaaactgaaacctcaagtaaaaatggactcaaaactcagacgcattactaaatggcgaagtacntngtgtgcgcaaacaatacaaacaaaacctattgttacacccattcgacaaatatttcaaccaaaaaacagaacgtgaccttaaaagtgagacaacttctgtaaacgtccacacgcctcaatgatagantaataaagccaaccaattcccagttcccataaccccaacccaacc

SEQ ID NO: 18 - DNA, 359 bp, Nicotiana tabacum
  plasmid a20-1-3
ggaataaagattaacataaatgtgatccccgaaaggtaaatacaaggatgccaatctctactaacatgaaatctctaatctctatttctcatgtccaacctcgtaaagcatgaagtccaaataaggcaagggaaacatttcattcatagaaacatgcagaaaagaatttatccagagtaataaaaactattaacctaaaacgtcataacaaaatgagcctggaataataccctacagcagtaaaacttaacgtccaaaaacacaacacataaaactcaaccacatcttgttctgctggtggagtaaagtaaaaaccaaaaaactaaaagggggggttgagttaaggggcttcatcatta

SEQ ID NO: 19 - DNA, 399 bp, Nicotiana tabacum
  plasmid a3-2-2 ; homology with L12 (60S) ribosomal protein
aagaagaccaaaaacattaagcataacggtaacatctcgctcgatgacgtcatcgagatcgctaaggtgatgaagccaagatcgatggcgaaggatttgagtggaacagtgaaggagattttgggcacgtgtgtatcagttggttgtacggtagatgggaaggatcctaaggatttgcagcaagagattgatgatggtgatgtcgagattcctctcgattgaatgcgaattatcaactgatngtaatattatgttaattttatgttattttgttttgaggatgtcatcttgaggatcattttgatataactatgacattctggaattttatatttggaaatgtagtttggatttgctttttctcgatgaagtgctttagcattgctttatgcgttttgc

SEQ ID NO: 20 - DNA, 287 bp, Nicotiana tabacum
  plasmid a8-1-1
gtgcaatttgcagtcactggcgcagatcgcagagaacttggctaaaagaaagagtaaattaacaactactcgtgactaattctgtgtttttttaatttttgtacattttctctcttttaatttaggttgtttgttgttttgagctgttagttttgaatgatggatagagtatttgttattattgtagattatgaagacccagaactgaaacttcatagattggtagatttcgatgactgtaaggttggttcttggaattgttacaacgtgactgtttgataattctg

SEQ ID NO: 21 - DNA, 284 bp, Nicotiana tabacum
  plasmid a8-1-2 ; homology with (chlorophyl)-geranyl-geranyl reductase
cagatgagtatgtgcagaagatgacatttgacagctatttgtacaagaaagtggcaccaggaaaccccattgaagacttgaagcttgctgtgaataccattggaagtttggtgagagctaatgcactaagaagggaaatggacaaactcagagtataagaggattaatagcattaatatttttcttgtaactgaagagtttatttctcaaattactctgtaaacacctttcatccttccttcaataggatttatgtaacttcatgatttgagttacatttcttc

SEQ ID NO: 22 - DNA, 287 bp, Nicotiana tabacum
  plasmid a8-1-4 ; homology with an early wound inducive gene
gaacatgctgattgcagcagttgaagaacgatatagagatgcagctctgtggagggacaagcttactcaactgcggtccaaacgaaactggatataacaggtgtgctttagagttgtctgagcaaaggactactgtgtatatagggagttattcatcggagccaatgtggtcagcatcgtcaaagatcaattgtagctctccgttaatatgtaaaataacttgtgaatatctgtatagattgtaatgctaatgtaaaacaaacaggtaaacttatggttcttggaca

SEQ ID NO: 23 - DNA, 344 bp, Nicotiana tabacum
  plasmid a9-1-2 ; homology with epoxide hydrolase [I]
cgttaaaagtggaatggtgaaagaatatgtgcctaatctggaaaccatattcttaccagaaggcagtcattttgtacaagagcagtttcctgaacaggtcaatcagttgattatcaccttcctcaaaaagctcatataataaactgcttgccagcgacgttgaataaagggcaacccagtgcacgaaactcccgttatgcacaaggtttgggaggagccggcatttgggtcttatttttcagagttgaatgttgatctcagttttatcaaacaataccatatcacattttcggcatatttctacttgtatgttgatcaataaaagggacgatggtttacgcgcc

SEQ ID NO: 24 - DNA, 255 bp, Nicotiana tabacum
  plasmid a9-3-4 ; homology with ISI10a glucosyl transferase [I]
aagagagtaatggtgagtgaagaagcagagggattcagaaacagagctaaagcgtataaggagatggcaagaaaagctattgaaggaggaggatcatcttacactggattgactactttgttggaagatattagtacatatagttttactggtcattaagttatgattaaaaaaaaagtagttcttagtatgatttctatactgtttttgtgctttttctgtatgtgactgtgctaatttaaacatttccttttg

SEQ ID NO: 25 - DNA, 216 bp, Nicotiana tabacum
  plasmid a9-4-1
cattaaacaagaattagcggtggganttgggcaagaaaattagaattggatctaccgtgtgtgctttttagcctattgaaaatcggattgcattttgctctaggcttatgatcttgttttagcttgctcctattggtgtttattttttantatgttttatgtattaaaggnaggattcagagaataaatacatattgtttatttctagttttgtca

SEQ ID NO: 26 - DNA, 212 bp, Nicotiana tabacum
  plasmid a9-5-9
ataagaagaaaattacctctacaatctttacttagaattgtggatgtagagcaaggatgcanagacccgagctaatatgaatttataaatatggattgttgatctataataagatataagtttcgatactttctgatattttgctatagaatttggagatgaatggtatctccagaactctcattcatttgtaaaaagtttttgattcttgg

SEQ ID NO: 27 - DNA, 199 bp, Nicotiana tabacum
  plasmid a9-6-11
taagcagtgacggagataccctttacagagagtgtgtgggtgtcatctaactagctgcttcataaaacatctnccttgtgtatatatctatatttaaattattttatatgtatatatagataatagctagttatcataatatantttaaatattgatttgagacaagaaataaaatctcaaaaccaacatattctttcc

SEQ ID NO: 28 - DNA, 178 bp, Nicotiana tabacum
  plasmid a9-7-1
gaaatgagagattgaatttcaatgantgcatttcaggaagagtactctgtgatgttcaaagtttgcagtgaattatcgtagtgtattnctagtggtggttggtncattacctttcccaaataagacatttattgtttgacatnccaattganaaatgtcattttgtatcgttctcttg

SEQ ID NO: 29 - DNA, 196 bp, Nicotiana tabacum
  plasmid a9-7-10 ; homology with LOX1 (lipoxygenase) [I]
tagaactttaattcaatataaaagtattaaatccangtgttgttattgtttctttatattcctaataataatagaaaataaaattttttatttttatttcaagggagttccagctacagctaaaggangtaatgctgtaggctcttctgttctgtaagtaattcatttgtatcaacaagtgcccagttttaaattg

SEQ ID NO: 30 - DNA, 197 bp, Nicotiana tabacum
  plasmid a9-7-11
gaagacaagaaaaactataggacattacgtaaatattgaatatagatagacttatgcgattgtgatgtaagaaacctttagaagacattgtcaaactccagcttctctaacttgtaagaaatgatcaagagtgaacctggcacagtcgatccgcaatttgttgctgttttgtcttcaatttaacactacgcttccac

SEQ ID NO: 31 - DNA, 340 bp, Nicotiana tabacum
  plasmid c1-1-3
aatcattaaggtttaaaagaaaagataacacgtaaaaacgcatcctttttacctttatcgtcaaatttcaaatgatgaattacggagaaaccgaatttgcaaactccataactctgctgctgttattctcgtctcagagagggagagacgcacaacgaacatcaaaatagcgggagaagctcggaaaaatatgttttcatatatttatataatttgaagtgaatttgttgtgttgaaaatttaactccctctgtggattgttattgaagatataattttttttcaatgttcgttttctgtttcgattattgaaagatagcaacagaaagattgtggctta

SEQ ID NO: 32 - DNA, 336 bp, Nicotiana tabacum
  plasmid c1-1-5
tgtatgatcgaggtgtaagccctcttcctgctgccaatgcagtagttggtctgaggagttgacaattgatgacaggtgttgacagttgatgattttctttcctactagattaaagtctaccttcactcatgtacatgataagcatttgtacagaacagttatggttctgtttataaaaaaagattaggtagtcttgacttgcatttctgtgtattttgaaagtgcagactcgctctttaacttctatgcgtgttggcttcttgggccttctccttcttgctcgtgattgcttcttataaaatttaagtaaaaatacatagcctggcattgttcttg

SEQ ID NO: 33 - DNA, 400 bp, Nicotiana tabacum
  plasmid c1-2-2
agctacganntgnctcnagggcnngcaantgcgncgngngantnatngcancnnnganntantgttnnanctggaacngantccangcaacctgtttctgtggattcttccacgtacctttggcttgttgatacatgtagatcgtattgccgtcaacacttaataacttgtacacgaaacagcttctgttttgaagtctttcccagtcaatggtcgatagcattaatcggctgagatggagcttagatcccaagagtagctgccttttagacggtttgacctaatcgtgtgttttgactctattatgataccttcatctgctgcactaagaaattgacaagtgcggtgaatttcttacatgaggaaatttcaactggaatgccttagtattattgtgttt

SEQ ID NO: 34 - DNA, 330 bp, Nicotiana tabacum
  plasmid c1-3-12
ggaatggatgatctgaaagcatcttaagtctaaaggaagtttgcaactcagttgagattcatccacactgagagaaacttctgaaacaaccatacttctgctttatcctgttgtaccatgaatagctgtagcagcagacaatgagctttttttaaagacatttggtttgtaacttaaaacggaaggaactggattgaggcaataagtgattctggagaatagtgttttgactcaaatatttaatttcattttccagatcatgatcacctcttgtgattttacatgtttaaggacttcaagtgaatgtattgttcagtaagtgttattacc

SEQ ID NO: 35 - DNA, 334 bp, Nicotiana tabacum
  plasmid c10-3-1
gagtaggatgctggtgggatggtcttctgttttacagaatcctttacagatctggtattcaagaagaccatgtaggatggtaggatgtcttgagatgaagcatgaattatcttacgccggaaattttaagaactttttgccatttttcatttacagctcaacagtttatatcgattagtagatttagagcttcctcattccatattctaatccttccaacacattatcctagtctgtctagtattccttttactgcattgggcaaactttgagctataattgtactggtcccaagcttcaaaagaatgtatgaaatgagccattcactcgttga

SEQ ID NO: 36 - DNA, 334 bp, Nicotiana tabacum
  plasmid c10-3-5
gnanagagngnaantttgggngganagntgctgttgcnaanccctantttcncccngccaantgnggaaaggaattaataaaanaagtttggattatngaacgtnggaagnaacaaaattagtaattcttattactagttattttcatttgttaacaccaataataactaatttgcttgtttggcttcatatctggatgctcgcttgtgtagcttattattgtcattgtttgtatgaataaaccaaggcgacgggcaactcttgactcttgtaaaaagtagacggtttctcagtgtagaagtcggagtagtaccattcctgaaatcttgtcttt

SEQ ID NO: 37 - DNA, 216 bp, Nicotiana tabacum
  plasmid c11-2-1
aatatgaaggggggtaaatccgtaaatataattaactaatcaaatatcgattacaaaattgtaagataattgattgaagaatatccttcttttgtacataattattttcaagattatataaaatgaaaattgatgtttgatcgagatgactttccattatttaagttgaaaatggagagtggttgtttcaatataagtattttaatctgattttct

SEQ ID NO: 38 - DNA, 179 bp, Nicotiana tabacum
  plasmid c11-3-1
aagtgttaagtaaaggtttccattgcttatccccggtatatttaccttatcattttctggttggacattaccgtgatagctagaagataatcatgttgactgagaaatcttatttctatgactgtaaaatttgttaaaaatgagaacgagataagatttcctattccgaagcacatact

SEQ ID NO: 39 - DNA, 182 bp, Nicotiana tabacum
  plasmid c11-3-3 ; homology with caffeoyl-CoA O-methyltransferase 3′ [I]
ggaggataaaatatcatcttgtaaataaactttactcaagccgaatgagacaaattttaagtatttgttacaatttcagaagtacaatatttgaaatacaaatatatagaaatattaatagcgataatagtcatgagatacaaaatatttattcacaaatcaaaagaaaaacaaaggtagtt

SEQ ID NO: 40 - DNA, 441 bp, Nicotiana tabacum
  plasmid c13-1-6
catcggatggaggacaaggcaagtgaaggggacagcaagaaacctcagagcagctcgaatagacagactcccacttcaaatccatttccagcttcttcgcaatctcctccaattgccaaatccacaagtaataaaagcaaaagcccgctgcctccatctttgccattgatatcagattcaacgtcgtcatcgtcgcaatctcctcctatagttgccaaatccacaagtaataaagttacaanaccgcaacctccatcttcgttgatatcagaatcaaattcatcttagaattcttgatgcagaatggccgtgctttatttgattcaccagtgattcttttgctcgatgctacaaaatactagtaattaactaccactcgagaagccttgcaaattttgtatacacgaatgcattcaatgaactgggatcgaccttctttgt

SEQ ID NO: 41 - DNA, 340 bp, Nicotiana tabacum
  plasmid c13-2-1 ; homology with L19 ribosomal protein
agggaccaggagagaggccagttcaacctgcagctccggctgttgccgcaccagcccaaccagctcagggatctaagaagtcaaagaagtgagcatgatgaattgtaaggagggtgccaagcctgctttttgttcttgctagtataacagtttagcatgtttgatctgttcccttattggtcttttaactttggaagacaacgttacctgtacgaatttggaagctggtttaaagttttgataccttgtttctcagtgataccttttactcatgttttgattatatattcaacttagttgttttgcgtcgcatggaatgtagtgagtgagcagctatttg

SEQ ID NO: 42 - DNA, 184 bp, Nicotiana tabacum
  plasmid c13-3-13 ; homology with 23S 4.5S rRNA genes (chl)
ccagagacgaggaagggcgtagtaatcgacgaaatgcttcggggagttgaaaataagcatagatccggagattcccgaatagggcaacctttcgaactgctgctgaatccatggacaagtaatgagacaaccatcttgctgtatattataaagcataagtaataatccattcttatagtgagtt

SEQ ID NO: 43 - DNA, 186 bp, Nicotiana tabacum
  plasmid c13-3-6
gaagacaatacaacattaatcacctttgcctctgcgacttagagacaattgaactactgcattttgcttgattttctatgttgtatcttgagtataataacgtcgtgagtgagtttatatttgcaaaggatatccagtccaatccatgcttgggttaaatgtatatttgccaaaaactttctattc

SEQ ID NO: 44 - DNA, 549 bp, Nicotiana tabacum
  plasmid c14-1-60 ; homology with a glycolate oxidase
ccttcaacaattcatggctcttgaagaggttgtgaaagctgcacaaggccggatccctgtattcttggatggaggtgtccgccgtggaactgatgtcttcaaagctttggcacttggagcttcaggcatttttattggaaggccagtagttttctcattggctgctgaaggagaagctggaatcaaaaaagtgttgcaaatgttgcgcgatgagtttgagctaactatggcattgagcggttgccgctcactgaacgagataacccgcaaccatattgtcactgaatgggatgctccacgtgctgctcttccagccccaaggttgtgaaaatgtacctcaagtgtcaaattgtttgatcaaagcaaagtattgcttcactgtttcagaagcttatattttggttttgaatacttgtttctgtttaatgagtttacgaatatgttaagcttttctcagtaatggaaaactgataaattctgataaatggccagatatgcctccatttgtacatcctctatttctatatatcatcatattgtgaacttttc

SEQ ID NO: 45 - DNA, 49 bp, Nicotiana tabacum
  plasmid c14-2-10
attgctatacttttccaagtttgataatatgaaaagacatttctgtttg

SEQ ID NO: 46 - DNA, 553 bp, Nicotiana tabacum
  plasmid c14-2-15 ;  homology with L35 (60S) ribosomal protein
ggggaaaatcaaagactgagcttttggctcagttaaaggatctgaaagcacaacttgctctcctccgtgttgctaaggtcactggcggtgccctaacaaactctccaaaattaaggtggtgaggttgtcaatagcacaagtattgacagtgatatcacagaagcagaagacagcattgagaaaagcttataagaacaagaagtacttgcctcttgacctccgtcccaagaagactagggccattcgtaaacgtcttaccaaacatcaggcatctttgaagactgaaagggagaagaagaaagagatgtactttccaattagaaagtatgccattaaggtttgaattgatccaacttagatagtttgtgatgttagagcaaagctgaggatcattatttttgccattttgcaatgttatattttgtattactactattattgcattatgaagttggagttttgttattttgtttgccttatgcgtgcaacttttatgcatgatcctgtctacacttctttttctacacttttgatcgagtgtcgtgattattgt

SEQ ID NO: 47 - DNA, 311 bp, Nicotiana tabacum
  plasmid c14-3-4 ; homology with L25 (60S) ribosomal protein
taaaaggaagattaaggatgccgtgaagaagatgtatgacatccagacnaagaaagtcaataccttgattaggcctgatgggactaagaaagcatatgtgaggttgactcctgactacgatgcattggacgttgccaacaaaattggaatcatctaaantagtagttacctgtttagaattttacgagaatttaaaatcttggattgagtttttagatacacttgaatggaagtgccttctatttttcattttgaattttgtgttttggagacatgttttgttccgtataagagaaatcaacttttatgct

SEQ ID NO: 48 - DNA, 272 bp, Nicotiana tabacum
  plasmid c14-5-1 ; rice genomic homology
actgggatagtcaaattattgatcatgaagatgggccactcgaaagggagaagcttctgtttgcagtgaaatcatattggacagcgccagctgctcaaggatcttaaactacttaatcccactgtttttaatctttcttacttcaaagtctaatcatattgctaatcctctcttttattctttcacatgttaagttctagtattacttgcaaattgtaaactctaggattttaatgattcttcagcaactacactgaagtaatgagttctgt

SEQ ID NO: 49 - DNA, 270 bp, Nicotiana tabacum
  plasmid c14-6-11 ; Arabidopsis genomic homology
ggaagattatgctggcgatcgccgatggacttggatcatcgccgattcaaatggttcttgatgatagtgaccagaatatgatcaaacaagctgccgatctcgaagcttctaagcgtcctgcctaattaattataactggtttccagttctctagcaaaataagtcctttttttattgtttcaattttcagtcatgtcttgtttccatgctgtgttctcaattctgtaattttacatacttatatacaaatgaaatgtaggacaactttat

SEQ ID NO: 50 - DNA, 193 bp, Nicotiana tabacum
  plasmid c14-7-4
tcaccaaattggcttgtnnacttataattattgttagcatataaaagaataactattgtcatattacatttttccctaatgttcaatgcctttttagttttcaacaaattcaatgttttttggttcacttgtttgtgagatgattgcaaaatcatcaatgtaatgcagtctatatttgaacgaaattcattga

SEQ ID NO: 51 - DNA, 203 bp, Nicotiana tabacum
  plasmid c15-1-2
aagaaatcctgaataacatttcatttgggaggaggtattatatagttaatggatttggggtttttttgccagtaaaattgtgttcaacatttaatagaactctgctgttgaaggggtttgtttttatatgattagttactgtatttgtattcaacagacaatattaattgaaatcaaatttctgcgtagaccaacttctcttt

SEQ ID NO: 52 - DNA, 492 bp, Nicotiana tabacum
  plasmid c15-1-4 ; homology with CBP20 (pathogen and wound-inducible antifungal protein) [I]
ggacctcgtggccgaaactcttgtggcaaatgcttaagggtgacaaatacaggcacaggagctcagaccacagtgagaatcgtggatcaatgcagcaatggcggactagacttggacgttaacgttttccggcagctcgacacagacggaagagggaatcaacgtggccaccttattgtgaactacgagtttgttaattgtggtgacaatatgaatgttctggtatccccagttgacaaggaataagaagctatatatggccatgtttagtctttgacggcccaaataaaagtaaaaagaacgatatgtaaaaggaaaaagaaaataaagttgctttgatggggttaggcaattccaatatctattcaagaatgtctttcgttttgggaagaaagagtgaantgtgtattatctttgtgattttgtatgcnaatattgtgatttttaaacaaanaatcncntgggacagtatttgttggtctccttttgaac

SEQ ID NO: 53 - DNA, 201 bp, Nicotiana tabacum
  plasmid c15-11-2
ggatcatgaggtctatcgagtgaaggcacatgcgatggcgagcaaaaaaaagcttttgcccatgtctagaacacaatgcggatacatttgatggcccatctgaaaggaactatactgcatccaagctgttaatggccataatattttccaatatcatgacatttcttcactgttattggataaacaagcttgagatctact

SEQ ID NO: 54 - DNA, 199 bp, Nicotiana tabacum
  plasmid c15-11-4 ; Arabidopsis genomic homology
agttgtacaccaaacttatccataagtttgaaaccattttatttccagtttacatgtactaaattatcggtagatttgcttatatgtattgtacagtagttctaatggaaaggttgatgtcaatatctccagagaggacagaatgacgaacaaactgtaggtgcgagaatattgcttctaaaacataaagtttcccgtt

SEQ ID NO: 55 - DNA, 431 bp, Nicotiana tabacum
  plasmid c15-2-8 ;  Arabidopsis genomic homology
gtcgcacaaaggcttccgtggatacaataccatgaagtacccaatgttggacatttgcttattcatgatcgagccgtgaaggaggttatctggaagacattcttggccggagagaaagagcagatagtgtattcttaaacgggaagaaggagatttagaggttcctttgtaagaagacacattctgtgtcttttactggtatatcctattgcatacatattaatcatatataaagttcgtgagctagtagctcaagttttggaacttcggtggataatggtttgcccctctaccctaactgagaaatcctggggagacgcaagtttcgaaactcgatggataatggatttgaccttctacccttctttaagacggttttgtggtacttgaatgtgcatttcggtttaaaacgttttaggtgtggccttgtg

SEQ ID NO: 56 - DNA, 446 bp, Nicotiana tabacum
  plasmid c15-3-4 ;  Arabidopsis genomic homology
aagaggaacaagtcatattgatcgctagatttngcatttaccgtgtggataaaatcctgtnggagtataatttcacttgggacgatgtactgaatttcaggctctactttgcaagtagtcttaatatccctcatagaacattgcctcgaatcttcactgatgtgtttaatgaatttgctcagatgagtcagagagttagcgtaaatgccgagcctatcttaaatatcgttccagtcttgggtgctgggaggtctttatcgaccttggatgatatattcacgtgtgaattcatcgctaggaaatgttagatctcatttaaattagggaattatatattaaatgttgagaaaaagagagttttgaacttgaacaaattcttataatgttattgccaacccaattgttgcaaattacacttagctttacaggaaatgaatatatgaagt

SEQ ID NO: 57 - DNA, 247 bp, Nicotiana tabacum
  plasmid c15-6-2
gaaccaagtaaaaggcctgaaatggaaaggaaaacaagcaatcacaactagacaacttcaacatagaagtgctttactacagtatttaaggacaaaatcaccaaaagctaatgaaaaaactggaggtgtttgagcttcaacactactctattggaaactgttgtatgccgatactatgattgtgttttggataatatttttgtggtgcaagttatgatgtaatatgatgtaaactattaaagcgtgt

SEQ ID NO: 58 - DNA, 325 bp, Nicotiana tabacum
  plasmid c15-6-3
accgatcaagtacctaattagagttccaaatgctgcttaggctttggtccaacaaggtcttgttgttccaggcatttaactcctttttgtggatatcgattctttatccgcctgtgagtggatgcttctgtttttgccatcttctggaaagtttagttgactgtaaaaacagctaaactgtaaactaaattagcagaggaaatctgccgccagatatttcaacatgcaaggatataatacttgtcgagaataaaattttcagcttctatggccttttctgtgatactttcaggaaaacattctatcagaaaatacatacgttctg

SEQ ID NO: 59 - DNA, 235 bp, Nicotiana tabacum
  plasmid c15-7-1
gttgatgatgtgaagctcttgagtgtcaggaaccctcgtcgattcctctgagtcatgtatttttatgtaaaacgatgaattttcgagttatagtatgagtaaatttggttgtaatgaagcaaaaagaatgtggggagttctgtttctcttagcttgtttactagtagtgttttcatatgagtatgtattatactaatgtctaatgaaaggcaaagaagtatatatattttgattg

SEQ ID NO: 60 - DNA, 307 bp, Nicotiana tabacum
  plasmid c15-8-5
taatgagcgtgacggaccaaatttagtatatagatagtacatatctttcgcattctagtacaatttatacccatacaagagtatacatttatgttactccatacaaatgaaagttaaaaaagttattgaatgtggaattcataatcatagggacaagcgatgtgaattctctatgttttgatgaacgacttgtatgatatgcttccttagaatacanaaattaaatatatttattgcnaaaaaaaaaatacntgactcanaggaatcnacgagggttcctgacnctcaagagcttcacntcntcanc

SEQ ID NO: 61 - DNA, 342 bp, Nicotiana tabacum
  plasmid c17-3-1
aagaatacaagtactgcatgcacaagcattccctngggcagagcttggatgatattaaaggttccttcgagtggtaaattggcaaaatctgctagcgtggcctgtgtacncctgcatcttttcccattaacaacttcctgttgtatgtattgtgtcnatcgtgtggatgctcattgatttgtactaatctgtaacgaagtgcaactttcagagattaaggttttgttttccatttcngtcccntggggtgttccggaacaactatggttgcttgtaaattcctctgatcttgacagtgggggcaatattcttacaaatttatttcaatttcaaccggttata

SEQ ID NO: 62 - DNA, 287 bp, Nicotiana tabacum
  plasmid c17-3-5
ataatgacgtgtcataaaaaatgtgatgtggatgacgacgtgtcatccacantgtgcatttgaagaacacagaggggtttaaagtagtgtgtttttaacaactacgagtgncttgataaaagcttgtggagtataggggccgagatgacaaatcaggacaagtaaaggtatttattaggctattatgccttaattatttataatttgcttaaacaatgtttttaaaaaatatttacagctattnacttgtatatcagacctttacatgaatttagcttattgttttt

SEQ ID NO: 63 - DNA, 211 bp, Nicotiana tabacum
  plasmid c17-5-5
attactattgagccttagactatgatggatatctataagaagaacaagcaaagcttgggtcgcttatggtggcctttgtgatttacattttactctacttcgaattttcaattaatttgattatattcttttgattagtttagttctatacttaacttgggattgttgatttactttgacctcttcacttagtattctcacttagttattg

SEQ ID NO: 64 - DNA, 211 bp, Nicotiana tabacum
  plasmid c17-5-8 ;  Arabidopsis genomic homology
attgaagaggattggggaaattcctgctgttgaggagtttgtttaccttaaattataagaactgtttgatttctgtctgaattcgctacaaagcaaaattttgatgatgttatttgtttaccagtagtagtctagtgcaggatacaaaaataatttggatgtgaaattagaagtgtagtacatttggttgtcaatttgacaatctttttgg

SEQ ID NO: 65 - DNA, 187 bp, Nicotiana tabacum
  plasmid c17-6-2
gatagtctattagttacccaaacctgctccgtatattttgcatattgtcaaagtgatctttcaggtacttcgtgattgttgtattcattctaaattttgcgatcaaaatagttcatccttagtgattgtacaantaatactaaaactggcactatttnggtttgaattcacantttctcacataatt

SEQ ID NO: 66 - DNA, 382 bp, Nicotiana tabacum
  plasmid c18-1-2 ; homology with DNA-J domain containing protein
cttgataagaggatggcaaacattcaaagccgcacctcgagttcggaggtttnatcccggtggtttgaacangttatgacaagaagggaagcagcattaattcttggagtcagagaaagtgctgtcctggagaagataaaggaggctcacaggagagtaatggttgcaaatcatccagacgccggtggtagccattatattgcttccaaaatcaatgaagctaaggaagtcttgttagggaaaaccaagacagctaattccgctttctaattcaccattttgtttgcaccttccttcttaacagcttaattgtccgtatacgtgtaacaaagtgaatttgtatccgtagacatgttactatcataatttaggagacttcttt

SEQ ID NO: 67 - DNA, 340 bp, Nicotiana tabacum
  plasmid c18-2-1 ; homology with CCT (chaperonin containing TCP-1) beta subunit
aatatctgagtcgttcaaagtcaaacaggcagtgttgctctctgccactgaggctgctgaaatgatcctaagggttgacgaaatcatcacttgtgccccaaggaggagagagggaatgtaaaaacaatattggtcatgtttaagctgttgagatgactcgtattttattatggtttgagaatttgagatggtaggtgtgggctgtaaacgagtcaaatgatagattgctattggaaccatgctaaagtgcactgcgctgagtagtttcttttgaggagcaaatgttttggtttgttttcataatgtatgcatgcttctatagaaaacatttgttcgatac

SEQ ID NO: 68 - DNA, 336 bp, Nicotiana tabacum
  plasmid c19-2-11
aaataaggttgcggaagcaaacaatccaggacattctgctggatcattggtataccgtaatgaaggtttngttantttgtttctgtggcattgttcaaatcttttatcagtnctccgcttctatagaggcaaaagggaatcctttctttcagcatgtacctgtaataatttgtaaaaataaaagttgataagtcatgtagctagctgtgttaatagaagaaagagatgagagtgagatttagtatagatgttttatctataccttnctgtggtatgtaggcttttactgctcanctcatacctcattgacacatctaatcaaattattccacttct

SEQ ID NO: 69 - DNA, 338 bp, Nicotiana tabacum
  plasmid c19-3-10
caggcaactaataccaagccattagtttctcattatgaaaaactttacaaagacaaaattacncanaactacaagccaaaaaagctcaacatagtaactntgatcaaatgatcatataatatttgcagccttggacacacctcagcaacagaatggaacntcaacaacactaanaanttgcacacctaaatccaaaacaaaaagactcgactccgtatcanaaantanggtttacntgaaaatgtatgatggtnancaacactgaaactgtctaacnantataanttcncnctctcaanacaancnttatctctgttcgttnanccgtttggttttat

SEQ ID NO: 70 - DNA, 323 bp, Nicotiana tabacum
  plasmid c19-4-19 ; Arabidopsis genomic homology
actaagtttctgcatttggcttgatttcttatcaagttgagacaatatttgtcattacaaggcatttttagtaccaaaaaaacattagcagtaactaaaaantatancttctggtttggggggattcancaatttgaagantctgttcgatgantttacaagctttcttgctcctaatctccactctcatgctttcactcttctcaatcttatcgtaagattccttcattttcagagacctcctcaattttgtcttcaagttcatcattaatctctcaaatcccatcatctccactctgtatttcttctcaatttaattgcct

SEQ ID NO: 71 - DNA, 326 bp, Nicotiana tabacum
  plasmid c19-4-22
taaaggatattgaaaagtaaatcctgcaagcacatataaaggtatgtttctacaaaaacataaatcgtataggtagaaatgaaaggcgggctgagagggaaagtgcagcanagtgatctcctgataggacttctgaaccacatnctacgtnggctttaaagcactcaaagccactactggagaaacagcactctccacttgtatctcaggaatgcactataagaaaatctantatactanctggacaatataataggtaggtatttaagtggaaaagggtaaagggacaagcccattatctaccatgttttgaactgcgcacncgg

SEQ ID NO: 72 - DNA, 256 bp, Nicotiana tabacum
  plasmid c19-5-1
atatacatctggagcaaatcacganttttaatacaaaactcaccctacaaaacatggantcnccactgcatcttaggcatntggacagcaanaaaacaagcaanttgtttggccgcctncactatttacatttactctattttgaattttttaatcaatttgattatnttatttggttattttanttctacacttaatctgggattgctgattcagtttngacttctttacttagtattctcacttcgtcactggc

SEQ ID NO: 73 - DNA, 257 bp, Nicotiana tabacum
  plasmid c19-5-4
atttaattgattagcggaaaatctncttttgtttnggtttatattgcacattctcatggatatttttactatttgtttcatagtttaacatcagcaagtgctttcttattctggtatattgacgccaatgtantaggctttgactttcttttaaacattgttgttgttgacatctaaaggttctctaaatttgaatttncactcttcaatttgcttcctttgaatgcaatattgctcgtcagctttgcatctttgtg

SEQ ID NO: 74 - DNA, 242 bp, Nicotiana tabacum
  plasmid c19-6-3
caaactaggatgtctgaaagactgaaagcgttagaagtaaataagtactcatttacagcggctgggtgtnacataccaaaacaaaacattcaacaagattgtatccaaaagaatacctggaaaaattacaacacttgggaactgaanaaccttanctgaccccagaaaaccattaaaggtaatatagcgcatctttacacggttgtgaanatcacaaaatatcctcaatttgttgcctaact

SEQ ID NO: 75 - DNA, 257 bp, Nicotiana tabacum
  plasmid c19-7-4 ; homology with putative translation initiation factor 2B beta sub. NIFb
ataaactatantaccatttagttgttgataatacgaatgaataaaccattcgacaacttaacttttcagtcaacaatagcatacgtgttgtctaataataccacaaaggaaaaccaccatcaagtagtactctgcatatccgaaatcacaaaactccagcacaaatctaatctcanaatcaatctacaaactccaaaaatcgcgatgctctcttcatctgtttattgcagtcagtataatgtaggtgcaacatcttg

SEQ ID NO: 76 - DNA, 384 bp, Nicotiana tabacum
  plasmid c2-1-10
gtgcagtaaactgaataggttgacagagctagctgccagatgactcttcatgcggtagggtttttcttatattactgccatacagtattggagctggagatatcaagaccgtgctagctctgctgattagttgtccgtatagatgacagtgatacataagctgacttggaatccaagtatctggtctaccacaattgattttctttgggatttactcacaatattcttaaacgatttttgccggataaatgcaatattcattgattgtaatcaatcactacaaggaggatgaagaatatattcttaaatgatttttgccagataaatgtaatattcatctatatggatagatgaattcttgatcaaatgtaagttcatgtcgat

SEQ ID NO: 77 - DNA, 181 bp, Nicotiana tabacum
  plasmid c2-11-14
tgatgcgcatatcaaaactaattattatccaagccaaagctatcctttgccagttgcttgataacacatatcttttgtgcttgattttaaaatacatgaggtgtatttgccgttgagtcatattgcagcggtgttcaatgtaatttacactgatacaaaataaggtaatttgtatattgtg

SEQ ID NO: 78 - DNA, 182 bp, Nicotiana tabacum
  plasmid c2-11-2
aggaaatactgcatcaaacggacaacaactcgatgcaggtgaagaatcctagtgctgtaattgctaataacaagcacatagtttgtctgctgtctttttactttaatattttcccctttgaagttgttggaatcgtattaattttgttagttaaaggcggatcaatcaatatatctttcctg

SEQ ID NO: 79 - DNA, 359 bp, Nicotiana tabacum
  plasmid c2-2-1
aacgggatactgaatggtaggacggcctcttcttcgaccactagcaaccatgtagccgaccaagttcaaagatgaaacatactgtattttgccagtggacattctttttgtgtggcttatccttataggtttttgttcattatctctggtattccttgtcaaagtacattatgatggcagacctctttagagagatcctcaaagtttatgtgttgtttatttatatcattttttctcgatagttaaatattaggggatattcttctttcggccatttgattttggttgaaggtcttgaatgtcgcaagaaatagctcagtttaaaggagttgatgaatgttctctccttctctgccgcc

SEQ ID NO: 80 - DNA, 356 bp, Nicotiana tabacum
  plasmid c2-2-3
agaaatatagggtaaggctgcgtataatagattcttgtggttcgacccttccctggcacccgagcttagtgcaccgggttgcccttttatttcagaagatgtatattatgaactcttggtttagattgagttcagattattttttaagaaattattttttagcaaagagtaagctcactctttgttcttattagtaataagtttgttaagttatcctttcacaaatgatatacagtattggtgtgaggtgtgtgagggtcatattcttgtgtattaattgttgcaatgcaacgtgtaattgctcaattggccagattggttttctcttcttaatgctaagcactacttgttatcat

SEQ ID NO: 81 - DNA, 338 bp, Nicotiana tabacum
  plasmid c2-4-1
gtggtttccgtgaatcgtgacgccaaatatcagttagcaatggtaactaactccatggcaacatactggaaatgagtgtgaaatctgaatttcagagttggtgtgacttcttcttgtatagctggtggttgttaacttgtcctagattcactctcactctcattggtgtggtccctgtgctagtgacgggtcttattgtggctctttagagttgatgttatatttactctacctatctgttgaagtttatccaattggtatactttttttgggttgttttaacaaagtgctattcgaatttgtaatttcaatttcgatcaaaccaccttaaatctgct

SEQ ID NO: 82 - DNA, 336 bp, Nicotiana tabacum
  plasmid c2-5-6
ggtggcctaatttgcagttttgatttagtgtcatcattagctattttctggattgaagttaaatgccggaaatctgtttgtaacctcaatcttcaacaaatcaattgaaatatcacttcaaggcacttcaggtcctccttgcacgggttgagagcttccaacagatttcggagattcactaggtagctgcttggcattcgcagcccaatgcttctccctctatcttattttctcctattttagttctgtaatagactatgtagactctttctgttttaaatcggttagtagatattcatgactggtgacaccccgttgtcgggctatgtctatttc

SEQ ID NO: 83 - DNA, 256 bp, Nicotiana tabacum
  plasmid c2-6-5
ttaaagaatgttttgtctaatcttgtgctggctttaatgcacgtcaaagtttgctgtcatcccctggcaatagcggacaacaaatctgccagctactgatgctgatgggtatttgtttaagtggagaagtaaataggattttatatctaatattattgcctttcatagttctcagagtatatgtgtagaacaagcacagctgcaaattgttattactaattttatggtggaaatctgttgaaagttattttctttt

SEQ ID NO: 84 - DNA, 254 bp, Nicotiana tabacum
  plasmid c2-7-1 ; homology with patatin 3′-strand
atgatgtcggttttgcattgtggaaatgcaagttttactttggcagattgctccaagtccttagggggtgatggatttcccctacaacagaattactatttttcctttctttttatgttgttttggcttagaaggatgattttatttatttaacacaaccaaaagtctacataatccttagcatatttcaaatttacatagagggatatttctattgaaatttatcccttaacgttacaagcgcttattcttta

SEQ ID NO: 85 - DNA, 219 bp, Nicotiana tabacum
  plasmid c2-9-14
gggaatacattgggtttgtcgtttgtttgtttggatgttagtagaccggcaagatatctagcattttgcttctgttaacatggacattatggatttgtaaattcaactgactacttgtacacgtctctctggacattcgggttattacttggtacaagttaataacacttatgctctctcttattttatgctttctgatgaatattccttttccctctg

SEQ ID NO: 86 - DNA, 337 bp, Nicotiana tabacum
  plasmid c20-1-4 ; homology with DNA-binding protein (pabf) [I]
gaagatgcgcttagacttggaggcagtgtagctacctacctctaatgtcaatttgttaggttaaagcaggatttgatattttgttgcacagtatgaagtatgttttagttctaactgtattagcagttgatttcgtcatttgataattaccttattctgctaatttggttaatgacaattaagggggagacaaaatcatgctcgtgggctatatgtactgttgtttgagtatgttgaatggatggaaatgcctttgttagatagatgtataatgccggcattatccctcatcaacagttgcctttgcaaatgtcgtaaaagcatttgaattttattg

SEQ ID NO: 87 - DNA, 337 bp, Nicotiana tabacum
  plasmid c3-2-4
aaagcaactccacgttagtgttataaaacgagtttaataaagtttgactctgatactatgtgaaagaatctaagcactaaaacaaaacctttaggcaatagtataacattgagatgtttcctttctaatttaaagaaggatagaagttcagtgcactctgctcacaagatgtagtacaaggattcttgaaccaaggattttgatggacttcatgttgagattggaaaactgaattcattactggagatcattgttcatggccctataaatttgaaatttcaaagatacaaatcaaattacttatatgtggcatacaacaagacactactaatacata

SEQ ID NO: 88 - DNA, 92 bp, Nicotiana tabacum
  plasmid c3-3-6
ggttgaccgtgcttaatataggcagggaggttgataattatataaagcacatctgaatgttaatccacgtaagaacttaatttgattgcttt

SEQ ID NO: 89 - DNA, 257 bp, Nicotiana tabacum
  plasmid c3-4-1
gcatagacttttttccaccatcagattagttggcttgcgataagagacgacttcttttagcaaatctatatgataacctgaagaatatagtaagaattaatctgctataaccagttaaatagtactaattacaactttttttttaaagttgtttgttaaacatttttcatgccattttgtttgtcaagtaccgaaaaaacgtgggttggctacaaaagtcttaacctggctagctagctacctgctactgagtatct

SEQ ID NO: 90 - DNA, 345 bp, Nicotiana tabacum
  plasmid c4-1-2
taatcaaaattggtaaaacaatccaaaccaaaaaaaacggtttnntgttgctcttgtttgaaatatattcgaatgttccttaatacctagcgtatgtaataataaaaatgtactcttgttgctcttgtttgtattgggattatttaattatatttgagatttataatttattaaaggctaatcgaatagtgttgactgatgtttggaaaatgtcatcagatatcaatgttggaagccatttagctcagtaaaattattttaactaaatcaaaagaataaaatactataggttggagtaaataagttgttaatggtagtgtttttctatttagtcatttgggatta

SEQ ID NO: 91 - DNA, 193 bp, Nicotiana tabacum
  plasmid c4-3-3
tactcacggggattaatctcatcacggtttcaaatggacaaacaattattttacatggagagtagagaccctccagcttctttttattgttagtagtagtgtgaattctcgtgttctcaatttggatagttatggtttctaacttatgtattagatcattttaacaagcagcacagagatcaaattgttcact

SEQ ID NO: 92 - DNA, 340 bp, Nicotiana tabacum
  plasmid c5-1-2
aaactagtggtttatttgtttcatcgtgaatatggagcagctgcaataatatcttcacaatagtactcattgactagatttgacacttcggatgaagccaaggcatcttcagagttttggattctacaatgtttccaagttatatctgcttttaatcgtttctgcttgtagcttaattgtcttttgatgctgtataccgtgtccaagtatgattgtagttttagggaatttcagattgcaaggcctttatttactcggatcaaatttgtaattgctagtcccctttttttgagaaattctgtatgtcccatttctttcttccaatggaactttcacttta

SEQ ID NO: 93 - DNA, 343 bp, Nicotiana tabacum
  plasmid c6-8-13
agcagaaagacaagtgtggttctggagccatgaaatcgcccgagtactctgcctcctcttgttctggtccaatgcagttttccactggtgttgctgtggcgtaagtcttgtatggtacgcaactcaaactaataaataaggaaactgtttatacagcttttggaaagctaacccaataagatttggtcataagtagatgggttatgttcagttttgagcaggcaatctctctgaatggaatgttgttcagcctgcccctattgagaggaagaggacttcttatttttcttaaacccatagacaagttcatctataaaaattaatcattattctttctttcctt

SEQ ID NO: 94 - DNA, 337 bp, Nicotiana tabacum
  plasmid c6-8-4
gataggtatcagatggaccttataagtgagaaaactcctaatgcaatcatctttacttattggaaatatttatagtgtgacagatacttggccaagtgctacagttatatgtactatttaatgaacaagttttatggtgtttggtatatgatgtaatttgttacttcagaatttattcttctgagtgtttcactggtagcatgatttacaagctaattgtatccattttctgagggataggatacagttagattgcttttcaatatctgatttgacactttgccctatgattcttgttttggaatggatacaagcaagcttattgctgttctgattg

SEQ ID NO: 95 - DNA, 294 bp, Nicotiana tabacum
  plasmid c6-8-9
atctacacgagcttcgtatgtgtaagactactggatcagattatccactgctctgataccatattaaaatcagtgacgtaatgaagcaattgaactcgaggtatgctccaattatggaaatggaaacttggcgaagaagccccaaattaggggcatgtgcgacannngagaagaagagaacttagaagtgaaagtctcaattgtattgactatgtaatgtcgtatatatcagtgttttaaaaggtgtggcgtaaggctaggcattttacacatacctcagcggggcgtaanata

SEQ ID NO: 96 - DNA, 338 bp, Nicotiana tabacum
  plasmid c7-1-2
caaaggactgtgcttcatagtggtgctgggagaggtnttgcagccactgacacatcaagcaccaacgaggaaaaggaattgaaagaaaataataaattcgatgtaggatcaaatttctatttggttgggttaattttantgaagttgatactgcaacaggagaatgacagtcctttgaaatttnaagttactattaatccaacaagagattgcgaatatgggaggtatgagatnatctctgtttctttaccgtcctttacatctgaaggcaacttagcataggagttcttaaatgtatcaaatatcaatattttcagcagagttcatttgttctttat

SEQ ID NO: 97 - DNA, 341 bp, Nicotiana tabacum
  plasmid c7-1-6
agtgaagggggcaagagaagaaaaggaaaagaagaaaatgtcagtcacaaacattcaagtgtttatatgtattcaacttttatactttctttcaaatgatttttacttttgcagatgggtgaaagaaaaagaaaagagtttttccaaactcgagacagaaaaagaaaagaaaaagcattcctctcttctgaatcttgattgcgtctttggtgtttgcggacaaatgtcctgagatgggtgaacttcacatggtcgcgtggtgttgtgctttgtgataaaatgtattgtgtatttatcatctttctactataaatcgaaattttattaagttgaagtcgtta

SEQ ID NO: 98 - DNA, 314 bp, Nicotiana tabacum
  plasmid c7-3-10
atagcatatatatgttgaagcccctgctcccaactcaaccccctccttttcttacagccattaatatattttggaatagctatttcctattttaggaaaaaacgaccatgtattgttcattgacaagtactttcataccctgctcaaagcaatatgtgttttctcgtacttggaagttaattttgctgtggaacaactcttgttagcttagtgttgtggggtgagctataactcggcctgtgtgatttgttacatttggttgagcattttctcttatataagaagagacagtgaggtgtctgtctcatggtcag

SEQ ID NO: 99 - DNA, 276 bp, Nicotiana tabacum
  plasmid c7-3-3 ;  Arabidopsis genomic homology
ggctgagaagaagaagcgcaaagctcagttgcaggaggccaatcggaaaaaaatgaataagagagtagagcgtaaaatggctgcagttnctagggatagagcatgggcagaaagactggcagaactgaagaagctcgaggaagagaagaaggcagccatggcttgatggttattgaacagagtttngatctgttaattttctctcttgtttttgagagtgaaaaatatattaatcccttatttaataggcacaattttcttcacacaatttttatt

SEQ ID NO: 100 - DNA, 418 bp, Nicotiana tabacum
  plasmid c7-3-9
acnaatnagaantaccacgtgnantgtcntgntacngtnataagngaagaggttcgatccngntcatcncaaatgncantggccccgtggnaagctcagccnngacaccggantgtttgcnngnggtnttattacagctaanntttatttctccaaangngataanagatngttctgtgannaggntnngattgnatccgccggagancagaaagtnattnttgcatcatanagtnggtnagangtgactcccntntctntgtcngnatatntntattggngggggntnttttagnattccagtncattccganatatagatcncanattncnatanntntacnanngcgcccccgcncgnntgtannncatnngggagatctcccanacgaggccggangtagagtgngaaaatctc

SEQ ID NO: 101 - DNA, 244 bp, Nicotiana tabacum
  plasmid c8-1-5
ggaatatgcataattttgttttcttttttgtttaaaagagttcaacctagttttatctgccagaagagagaaacatcaagatgtgagcatcagacaagcttataatactctctctatatagatttctacaaagcttatttttggtgaatgcttgtgttgtgtgtaatacttcaaccccatggaaatgctacgtttattagctcgtgctgtggcacccaaatgaatcttgattgtgtcatgttct

SEQ ID NO: 102 - DNA, 346 bp, Nicotiana tabacum
  plasmid c9-1-4 ; homology with Drosophila heat shock protein 82
gaagtcgaggaccgtgcccaacggtcagcaattacaagagtaaatgcagatgatgttcgggtcactgtatccgcacctgcagctcgtggagaagctaacaatgaacttatggaattcatgggtcgagtactgggtctgaaactatctcagatgactctccaaagagggtggaatagcaaatcaaagcttcttgtagtggaggatttgacagctagacaagtatatgagaaactcttggaagctgcccaaccttgagatggctccctgatccttttcttctttgtcattttttccatgtttgtaacattggatttttagtttcataaaattgaattcagttgtcttt

SEQ ID NO: 103 - DNA, 360 bp, Nicotiana tabacum
  plasmid g10-1-1 ;  Arabidopsis genomic homology
gaacgagaacaaaccatctcaaaagtacatcgagatagtgactgaagataattttgaattttggttcatgggctttgtacgatatgaaaaagctttcttgaatttacaaaaggctatttccatcacgaattagctagctgttaggcattagaatttttagggttttaaagaggattcataattctgtaattgttcttttttccttattaaatgttgaactggtagcatctaatctatgcttgttcatcattttcttttctctcaacggaagaggatttgagatttatgagaattgaattttgtagattctgaaatttaatgaatttctcaacatatatataagatttagaccaaagttac

SEQ ID NO: 104 - DNA, 556 bp, Nicotiana tabacum
  plasmid g12-1-21 ;  Arabidopsis genomic ABA-regulated gene cluster homology
ggtgggatttgactatgcatatcgcaaagcaatgaattcgactatgaaattcatcacaagctcaaagaacaaggcgtatacattttttagaacgactacccccgatcactttgagaatggtgaatggaatacgggaggttattgtaatagaacaggacccttcaaacaagatgaggttgacattggttatgtagatgaggtgatgcgcaaaattgaattagaagaattcgagagtatatcgagaacagaatctgcagacaggttgacaatgaaattgttcgataccactttcctttcgctgctgagaccagatgggcaccctggagtctacaggcaatatcagccatttgctaaagaaaatatgaacaaaaagattcagaatgattgtctacattggtgcttgcccggcccaatagattcgtggaacgatgtaatgatggaaatgttgttcaccagttgaaaatggtgtgacattagattttgattttgctcccacaattgtattgttcatctgcaaaagatggttgcacactatttttcaccattgtttcctctc

SEQ ID NO: 105 - DNA, 579 bp, Nicotiana tabacum
  plasmid g12-1-5 ; Arabidopsis membrane related protein CP5 homology
tattattcaagttggtatattggagaagtggaatcaagtagaggtaacagccagccgacgcgatgtgaagtgattctattccatcatgaagatatgggcatcccatgggaaattgcaaaatttggggtaaagcaaggtatgtggggagctgtgaggaagattgagcggggattccgtgcctaccagaaagctaaagcatctggcttgaaaatatctcattgtgcttttatggctagagttaatacaaaaattgatcgagaatacttgaagtcaatggaagatgatgaggactcatctgaaactgaattgcaagcttcacctgcaaaacctgagggcatgaacataccaaagctgattatcattggtggagctgtggcagttgcttgtacccttaatcaaggaatcttacccaaggtgcttttgtttaatgctgtgaaaaggtttggaaatataggaaggagagcatgtccaaggacatgacatttgattcatgcgtgcattgcgcatttgttttttccctgtttaagcattcacttttaagctctttatatatttaaaacaagcaagtgttattttgtc

SEQ ID NO: 106 - DNA, 358 bp, Nicotiana tabacum
  plasmid g14-2-4 ; homology with vetaspiradiene synthase PVS4 (sesquiterpene cyclase)
gatagcatggaaggatgtgaatgaaggaattcttcgaccaactcctgtttctacagaaattctcactcgtattctcaatctcgctcgtattatagatgtcacttacaagcacaatcaagatggatacactcatccagaaaaggttctaaaacctcacatcatcgctttactggtggactccattgaaatctaaaccattgagtgcttttttcatctcggtgatcgttttatttttatttttaaataaaggatcagaactgtgtttctgtgttcctctttatataagcaagttgagtttcctacttctgttcaaaccctgtgtttgttcttggcgtctgaataatataattttgtttgc

SEQ ID NO: 107 - DNA, 264 bp, Nicotiana tabacum
  plasmid g14-3-10
caaagataaagaaggctggagttgtaagacaggagcttgctaagcttaagaaggacgctgcttaagaactctttgattagtgagatttgtatgataggagttttggaagtcgttgtgttttgcttttagattttggttcattactggcaagtcatttggtttcatctttggtgtcattgaagactcctagaaatcaatttcccaatagttttcatttgnnttatgatggtgaacattctcttcgcagacacttcattttgttgc

SEQ ID NO: 108 - DNA, 211 bp, Nicotiana tabacum
  plasmid g14-3-22 ; homology with orf 03 A. thaliana
cttccatcaagcagggactggttgggggactttatggtgtggaaaccagcagttggtatggagaatagccaatcattctgggcaattttaacaatatggatagctttggttggagctgcactctttttgcaaaagtgaatcatatacaagtaaagctgtttattgtctagctttctattctttattggtatatatagtctgatgtgtattg

SEQ ID NO: 109 - DNA, 262 bp, Nicotiana tabacum
  plasmid g14-3-3 ; homology with sequence 161 from patent EP0953640
acattataataggatgtaaagaatgaagcaggaagcagtttcttactagaacttctactataattgtggatttatattgggttgttcattcagaaagctttgccaagtaacttagaattagtgtttacattttgatgtctttgttttgatattactaagaagaaaagatattggggaaaaaagaaagccagaccactgaatggcaggtctgatatgaaaactggccatgtatagaaggatatttcgtttatttcattttttg

SEQ ID NO: 110 - DNA, 265 bp, Nicotiana tabacum
  plasmid g14-3-4
gcttcaagtggatgatgatgatattaaggccatgattaaattgggccgtggtgatgaaaatggtggtggtgtcacctttgaaggttttctccaaattttgtctctttgatttgttgctttgatgacgatgataaatgtcagattaggtgaacaagttttggtttactttgtatttttcaatgatttgttttactgtgctgcttcatatgctattggctattccgagaattctatttgaaaacaaagaagaaaaagagttgttccg

SEQ ID NO: 111 - DNA, 260 bp, Nicotiana tabacum
  plasmid g14-3-7
atgaagaagaagagggcggtggtgatgactacattgagtttgaggatgaagacattgacaaaatctaaatctgaacgcaaagctgctgttactgaggtccgttataggcctttctaatgtttttgtggagctttttccataaacattgagagtgtatcttgtgtatcgtttgaagttatgtatcaaactttgtgcattgtgagttttgtattagatttatgcttccatgaaatgaatgcaatattctagctggtgtctac

SEQ ID NO: 112 - DNA, 469 bp, Nicotiana tabacum
  plasmid g15-1-37 ; Arabidopsis genomic homology
atattcctggaaacatctcaacttgcatcatccccacttcgtcaagatctaccgccaagtgtcatactgcaccatctttactcacgcgggcctgaagaactacaatcaccattgcaaagaaatagacttactccgacgcagtattcactctggatggattcacaaggggaggaccaaatctggaaaggtattaaagctactctggacgactatgctgctaaggtacggtcaagaggggacaaggaatttagtcctgtctatcctttgatgctagaaatcggctcttctttatctgggaatcgttagaggagctttgagagaatgcaaagctcaaatcatcttctcttggtatatgcccttccccatatttttgtttcaataatattgtcacagatgaacacatagcagaccgttatctatgtttcgtttagtgtcttactttctttatatattttacctcaattgattg

SEQ ID NO: 113 - DNA, 350 bp, Nicotiana tabacum
  plasmid g15-2-2 ;homology with ubiquitin [I] able to induce HR-like lesions
gttgatgtcgttgtgtcgtgttgattgactgtgtctgtttctggttgtggtcgtgatgtgctttgtctactgaggtctcaaagatgttctatgctatttctgtttgctgtttctcttatgttctctgttgtgaataaagattccgaattctgtcctaaaaaaaaaaaaatgaagtttatgtatattggaagaagcattggtgtcgtcaccaagtcccatttgatatatggctgtgtttttgcttggctaatttgtgtttaaactttctttctatctgtgctcaatatactcctgaacagactgatgtacgattttaaagctatgtatgtataaactctcttatcttttgc

SEQ ID NO: 114 - DNA, 345 bp, Nicotiana tabacum
  plasmid g15-3-11 ; homology with sequence 7 form patent EP0953640 [I]
gtggatgaagttaaggtgacccctgttgcttagaagtacacagagcttttgtaatggtcaatagagttttttgcaatgctaatttcatacttattaagctaccactgtgaggcaattgctgtattttacctatgtgattgctttaaactatgaattagatgcctgctgtgagacttgtgtactattgcttttaaggaagtgtggatctagttgaacttcctctcctttactatgtgcactttgatcttgattcttagatagtcaagaagtaatatataaaattgtactactatatttcaaatttttcatgtttcttgaaggatgaaatataaatgagttagtacc

SEQ ID NO: 115 - DNA, 344 bp, Nicotiana tabacum
  plasmid g15-3-7 ; Arabidopsis genomic homology
gatacatggaatgagttagtgtttgatctcatagggagagacttccagagtagacagagcaatgcttcataagaagaaggatccttaatgctaaaaaacattttttgtgcttctacagcacagctacgggaagattatttatctctctcgaatggagtttagctttttagttactttagatctcttgttgtagctggtgttgtaatctatgtttagatatccacggtaagataattcctaagttacacgaaattttcacaggtctcaagtatgtgtgcagggatatttaactaaatacaaacgttttctttgcaataaaatatttcatctgatttttccctcgc

SEQ ID NO: 116 - DNA, 301 bp, Nicotiana tabacum
  plasmid g15-4-1
tgaatgtttaatgttagaaagtgaattactctctttatgtggtgtctgaacatatgttcaacattactcttcaaattaccaataattaatagtgcgacaagttataggttataggttgatgaaaaattgtttccatcttgtaaattatagtgctaaatttatcacacatctgtgtgcacctatattatagtttctgctttcattgaaaatgagtttcaagttttctagtggaattggatatgtagtatagaagttggagggttgcttttcattcttttgaaagggtaaagcaaacttaagc

SEQ ID NO: 117 - DNA, 525 bp, Nicotiana tabacum
  plasmid g17-2-13 ; homology with wrky (zinc finger DNA binding protein)
aagtggatattttggatgatggttatagatggaggaaatacggacagaaggctgtcaagaacaacagattcccaagaagctactaccgatgcacgcatcaaggatgtaacgtgaagaaacaagtacaaaggctgtcaaaggatgaaggagtagtagtaactacttatgaaggcatgcattcacatcccattgagaagtccacagataactttgagcacattttgactcagatgcaaatctatgcttccttttgaaacgtccatcacttcaatgcctaaggcatgacactcaattagtcacttgtaaaatagtactacagtatattgtgtacatgcgttttgaacctagatgctatattttgaaataaaacgcaacttcattagggaatttaatttgatcattgtacaactaaaagtaatgttgctatttttttgtttttatcactttgtttttgccggagccatgncttcattttaactctttcttttagaattaacaaataattncatgttggagaagancgtg

SEQ ID NO: 118 - DNA, 225 bp, Nicotiana tabacum
  plasmid g17-3-2
gaccaaatgagcaaattgaagaaatgctggagatcaccacatacttccaggcaaagcaacctcaatttttgttaccaaaagatttcttgattaaacttttgaaagtaaacacgtgtgtgtagagaagtaaatgcaggcactgggatttcaatatcgtttcattgatgctggtacagtaggagattgaaactaaacattttcttgaagttcagtacgtgttcattg

SEQ ID NO: 119 - DNA, 412 bp, Nicotiana tabacum
  plasmid g18-4-7 ; homology with L18 (60S) ribosomal protein
attgagaaggctggaggagaatgcttgacctttgatcagcttgctcttagagcccctctcgggcagaacacggtactgcttaggggtccaaagaactcgcgggaagctgttaaacactttggtagagctcctggtgtcccacacagccacacgaagccttatgttcgggcaaagggaaggaagtttgagcgagcaagagggaaaagaaagagcagaggtttcaaggtttgaggaattgcgagtgtttgagtgcacgatgagagaatttcttttagaaggttttccctacctactttttaccatattagcttctttttcttgtcgaatttcttatttcacccctgtttctgtgacactccaacctatagccgattttgaatgcttttattatctattctacgaaattaagctg

SEQ ID NO: 120 - DNA, 373 bp, Nicotiana tabacum
  plasmid g18-5-1
acattatcaagacgaaggcaataagtgggcttactcattcttactgaaaaacggggctgtgaaatttgttgtaatcttcaagaatgtacttgttgccatcaatagaaaagcaaacaatattgtgttcagttacagccttgttgggtcttgctgagagttatttttctagttcctgaaagttatcttgcaagctatcatgtagctgtgtggtaattttcacaggtttgagctacagttgaagccagtaacatgtgttgatattatagctaaaataactaatgcttacctgcagtttccgtttgtgtggaataaggagaagaattgatgtgtaagcatggcttctgtgagttgactctattatctattgcattac

SEQ ID NO: 121 - DNA, 390 bp, Nicotiana tabacum
  plasmid g18-5-12 ;  homology with capsanthin/capsorubin synthase, promoter region
ggttgcaagggtgtatccgaaccctatttgcagaaaaattatactgtatatacaaggtcaaaattattttttctgtttatatagttagatgttaaattgtcttggctttttcgtgtatttatttctttatattttgaatcttcttggtgaaaatcctagctctgtacacacaaagagccgacatgctgatctctctctctctctggacggagagtcttctgaagtgattttgtgcttcttcagtgtgtttatagatcaatttagtgtctttgtcaaatggatttctaagtgaaaaaagagaaaaagtatttcaatgcgtgtgacctaccttgcataaactctgcatgatggatatacaatgtttctgcttgatatatgtatatgttttgg

SEQ ID NO: 122 - DNA, 381 bp, Nicotiana tabacum
  plasmid g18-6-12
tcttgcacgaggctggttatacaagggactcatggttgcttctgaatgacttcattaagatcctggaccaccctggtttgaagatggaggtagaagtaccaattgactagttacacctgcaatttcatttactataattcagatgtatctgtgtacaaggcagccgtgttattctgttttgttgaattcgcgcacctgcattctcctgctgttttttgttaaatctctttctttttccttcttttgcccccgttttatgtctgtttgcgcggcagggacagaaacagagaaaccgccgtgtaattaagataaaagctttcagcttattcagaagatcttgaatatgctataattttaatctctcacaaactgtgtatcttt

SEQ ID NO: 123 - DNA, 356 bp, Nicotiana tabacum
  plasmid g18-6-5
ttagagaaaaagagagaggaaaatcgtagaaaaatcttcaaaaaactgagttgagtaaaatttcaaaaaattttagttgtcatttctcttctggtctttcctttccagtcgatctcttcttcagaaaacaaaaaaaaatggttcaactttagttttgagtccagatttgatctcatttctttgctagagtttcgtttgctgttatttgctggttttttgctttacccgtggctgaacttccttcatctttatttctgctctctaccagctatttcgagctttatttgttaagtattctaggtacacactttcaaatctgtactgtttcttcatgaaaagggctgaaaattttgaat

SEQ ID NO: 124 - DNA, 293 bp, Nicotiana tabacum
  plasmid g18-7-5 ; Arabidopsis genomic homology
aagaaaagtagcaccaggggcttgtccttgttgtggaggaaaagtacaagctgtagatgtagaaggccgtttcagattttgctttctccctatttgctttaggttcaagaggaagtatctctgtactctctgttctaagcgtttggttttgtattcttgatctccctattttcctcttgtaatttctactctcaattttttgaacagcatcctataagtgtaattatttatttgaaatagtgtttgagagttgttcatttgctcaagaatatatgaaacttttgtagttgtgc

SEQ ID NO: 125 - DNA, 259 bp, Nicotiana tabacum
  plasmid g18-8-7
tgaagatgtagataaattgctggaagatataggggatgatgttggtgctgatgatggtgacgatgaaaactagaatgatgttttttttctcaagtaaatttatntcattgtatttcttgttagtttttctcttctccactcccctctgtttttctgtggcgcataggttgtacattgtaaaaatttcccaataccaacataatttaaggatgtaaaccatcttcttgctttgcttgtaatttctctactaggttgcttt

SEQ ID NO: 126 - DNA, 491 bp, Nicotiana tabacum
  plasmid g19-1-5 ; Arabidopsis genomic homology
ggttttaataagcttattggtggttggttgttcgagttttttggttactttaggagaggcaagtggtaggtggacgagttttggggttatatttcaaatggtagtgagttcaggatttgcaactctgttaatgcttcagagtcttgctgtgaacgtggtgttgtatatgtattgcaaggcatatcgtggggagctggcgtttgagatcgcggaggagtttgcgagtcagtatgtgtgtttgccttttgataatgagaaggttcctcatcttgtttgtgttgttcaagattgaatgtgcctaaggtcagtgagattatgttaggatgatgcagttagtagtttgaagaagtagtgttttgttttactcgtagcatgtatatagtttcttgtttgttagataaatgattgaagatgtgtgttacctgttggcaatgtgcatctttatatgtaaaaaaagcttaatacctgttatgaaattccctccnagtttt

SEQ ID NO: 127 - DNA, 485 bp, Nicotiana tabacum
  plasmid g19-1-6
taggaaatgacctttgcaggagttaaatcatataaatatttttttggactgcaaataatgataatttttctttttctaaccaaagcaaaataatatcatttgtgaaattcagtcggtgtacctgaacattattagtattaaaatggagaaatgagagaacacgtatggccactagagatattaaagctacctaaatatgacaatagatgaagcagaggacagtataatataattttcttttaactataacatacattgccccctttatagatcaaagtttttctactattatttaatttattactataataatcatctctctctaggcggctagttgggactatgctcaacttgcaatatttaattttgttttcatgttgttcctttttctggatgatgttttaactgtcgaaaaaattgagagctaagttgcatggttctgagttcgaaggattaaaagcaatgtnaatcaattggctctatgc

SEQ ID NO: 128 - DNA, 484 bp, Nicotiana tabacum
  plasmid g19-1-7 ; Arabidopsis genomic homology
ggaggaaagatctaggaatttttccgagtttgaacaattcttggttgatcgtttctaccgtcaatgaaggcagaaacagcggttttgaatccacctctcatctcttttgacaacaagagggatgcttatggatttgctgtacgacctcagcatgtacaaagataccgtgaatatgctaatatctacaaggaagaagaggaagagaggtctgataggtggaacgattttttggagcgtcaagcagagtctgctcagttacccataaatgggatatctgcagacaaaagttctactaatcctggtgccaaaccatttagtcaggaggtaagttgtgatgcacagaacggggaagaaggtcaacttgaaaatgcaactgagaaggatgtcatactgacctctgtggagaggaaaatttgtcagactcagatgtggacggaaattagaccctctctacaggcagttgaggatatgatgaacactcgtg

SEQ ID NO: 129 - DNA, 224 bp, Nicotiana tabacum
  plasmid g19-2-1
ttttttttttttgggtggcggaggaaagcgtgtggaaaaaaagaaagaaaaaagagaaccatagagttaaaggccagatcatgtctgctatgagtcatcatctgttgttggaagagaattcacttgtttaattttacttctcatattttatatcatgggatttcatgttggatggatggaccagtgtgtatgtcaaattaattcttattgcgaaaaaaaaaaaa

SEQ ID NO: 130 - DNA, 198 bp, Nicotiana tabacum
  plasmid g19-2-9
ccagtgtaattggactttgcgcaattgagagacaaggggttagaggtatatacgtgattgaagatcgtgatctatcttgttatctctcatttttttgagatttttctcttcttctttttccccaaatctgtaattgatgagattctagacagtgttagtgtataatcactagataatctatgtataatcagtttatcc

SEQ ID NO: 131 - DNA, 204 bp, Nicotiana tabacum
  plasmid g2-1-2 ; homology with 5-epi-aristolochene synthase (sesquiterpene cyclase) [I]
ggactccatcgaagtttgagctgccaattgttgctcatcttaaagaaacttcattcttctgtgttgagaaagtagttatatatgtttttttaaattgtataattaagttgttaggaagctggttttgcgattgtgcagtggacttcctaactaggacctccttgtaagaagtaatcttcaagtgttatgaattcacttgcattg

SEQ ID NO: 132 - DNA, 313 bp, Nicotiana tabacum
  plasmid g20-2-20 ; Arabidopsis genomic homology
tgcgagaaagaccaagaaatttgtattagagcaaaaaatggtgcttgggtgatttcgcgggtgacacgagggaaggagctctatatggtacttgagaaagccaatgagacccttctttatgcctctgaagctgttgaaaagttcagtgacaggtattgcagtggcgctttttctttgtaagagggaaactagattttggtattgccgagacacaggattcatacaaaagacatagctacatatcttatgttgttgttaattcaactttgtttgtactgtttataaataaataaaaacttgatcctctcctctt

SEQ ID NO: 133 - DNA, 315 bp, Nicotiana tabacum
  plasmid g20-2-29
ttgcaatgaactttgtaactaaggtgggctataaagaaggtttgggaacttcttatatttagttgtttacgagacaaattcgtgctttcctggtttatcaagaaaagaattggtcaacttaatgaagcatgtctccacactgatctatctattctgatttccagtgtaacagcttttttggccattacagtggttatttgatgatcactagcattatcatatctagtaaagtaaacacgtcaagtcaattgatccattcaactgtaactatgctgaattttacttatggaaaattcggaaaatactatttacttc

SEQ ID NO: 134 - DNA, 315 bp, Nicotiana tabacum
  plasmid g20-2-31
agaatatagctactacaaggtggttctcccagtagatcaactcaaagccattactccgtcaactatgctgtcaagaatttgcaaggtgcattgctgggtcatcattcgtagctagcgtgtcattttcttggtcatttcagatgaggtccgtgacactggtgcttgcttttgttgtagataaaattctgtaaagtatgcacatctgggtgattgattgttgcatacatgctaatttatcagcggtttggtatcttgtgtacatctgtttcctgaattttttattatcttttagtattactttggttggttcgattg

SEQ ID NO: 135 - DNA, 483 bp, Nicotiana tabacum
  plasmid g3-1-1 ; Arabidopsis genomic homology
attttgagaccagaagggaagctcattgtccgtgacaaagtggaagctgtaaccgaattagaaagcatgttcaagtctatgcattatgaaatccgtatgacctattcaaaggacaaggaaggattgttgtgtgtgcagaaaacaatgtggcgaccaacggaggttgagacactaactaatgcccttgcttagctgcttagcgtgtgtgcggatgctggttgtatatcattcgagaggctttcatgccacggtgactagatagtttttcgattaaattcttgttactgtattcttgtcaggctaccgtgtaccattccatagcaaaattagtgctattatcactatatatttgtggaaagtaagttttgtaatattatgtcattagttgtggaggaggtggacattcttggaattgtaaatgccattggtttaggacggtggtaaaaattcaaaaacaccagaatgaaattcgttttcagagcg

SEQ ID NO: 136 - DNA, 553 bp, Nicotiana tabacum
  plasmid g3-1-4 ; homology with ADP-ribosylation factor
atagcaatgacagagaccgtgttgtggaggcaagagatgaattgcacaggatgttgaacgaggatgagcttcgggatgctgtgctgcttgtgtttgctaacaaacaagatcttcctaatgcaatgaatgctgctgaaataactgataagcttggactccactctctcaggcagcgtcactggtacatccagagcacttgtgcaacttctggagagggactttatgaggggcttgattggctttctaacaatattgctaacaaggcctaaaccaacgtagagttgttgcgggttgatcctggatgcaggcgggtttttatctagttctttttcctttttttcccgaacattcccagaatctgtgtggttatgaatatcccttgaaagtgatttgcttcttggtaggacctattgaaatgtttttgtaatacagtggttggatatatgtaattgtttgtttagtttaaagtataatgctataatttgtaacagagattagatgtttgatgtttcattggtaaatggtaatggtatacttccctgtttgttccttc

SEQ ID NO: 137 - DNA, 501 bp, Nicotiana tabacum
  plasmid g6-2-13 ; homology with ACC oxidase
gagctctggtaattaacattggcgatgctcttcaaataatgagcaatggacgatacaagagtattgagcatcgagttatggctaatggcagtaataataggatttctgtgccaatttttgtgaaccctaagcctagtgatgtaattggtcctttggcagaagtgctagagaatggagaggaaccaatttacaaacaagttctttactcagattatgtcaagcatttctttaggaaagctcatgatgggaaagacactgttgattttgctaaaatcaagtagaaattagtggatctgctcgaagaataagaagtgcgcttatattaagctaatgtatttttctttcatgtatttttagttacgactactcagcaatttaaaaaaaaagaagagatagtctcatactgcaaagtataggagaatatttttgggattaattaggtgttcgaattttgtaccggataaattataattgagctgctgatattatggcaaatttagc

SEQ ID NO: 138 - DNA, 373 bp, Nicotiana tabacum
  plasmid g6-3-7 ; homology with ATP citrate lyase
aaatagtagagatcggttacctgaatggtctgtttgtgctggcacgttctattggtcttatcgggcacacatttgatcagaagagattgaagcagcctctataccgtcacccatgggaagatgttctctacaccaagtgaagacgctcccaatagcagcacgcagaaagtcgcctgcttcctatccagcattttatcgaaaagtgtttgtttagtcatttgttgtgatcattcttcttgttttctgctagtattttgtactcctaagaacttgctaagcatttctgtaagttgttataagagacaactcttttagtttcacaccaagagtttccttcaattcctatatatcaaagaaataacacattcattgt

SEQ ID NO: 139 - DNA, 301 bp, Nicotiana tabacum
  plasmid g6-4-4
gttgggggaaaaggcaaaaagatgaagaaaaaggcaatggaatggaaggaattgactgaagcatctgctaaagaacattcagggtcatcttatgtgaacattgagaaggtggtcaatgatattcttctttcgtccaaacattaagttaaataagttactacatcatttaatcttccttaaatttcattcttgtgttcttgtaagtctttttcatacttatttcccttcttactttcgttttgcattgtcacagtgtaaggttggaagcaaataatatatcctgcttaatgtcgtttggtcg

SEQ ID NO: 140 - DNA, 299 bp, Nicotiana tabacum
  plasmid g6-4-5
aggttatagatgaaagaccaatggctttagtaactgatgctgttgcgaatgaagccaaagataaaggctcaagctagaaattgcagtaatactgattttattgctgtcttctttaacattaccatcactaactagttctccatttttcttactggtgtatttactttcaagtattttatttgatgaggcgatatctcattacttttgtttttccagttgtttgctttagtgaatttatatgctggaaggatttgaggtattagatagaaagcatcttctgatttaacttcaattatgtg

SEQ ID NO: 141 - DNA, 356 bp, Nicotiana tabacum
  plasmid g7-1-1 ; homology with a A. thaliana gene homologous to MEI2 (meiotic regulator)
cagtggaggactcgaaatggaacctgatgatcaaaataatttgcttaatggtattgcaaacttaagcatgtcttatagttatccaaatggtgctgcaactgttgtcggggaacacccatatggagagcatccgtcaaggacattattcgttcgaaatattaacagcaacgtagaggactcagagttgaaatcgctctttgaagtagtgcttaacttaccagtttctttaaatttgcctctgttaattagctatcctttttcgtacttcctttattgcagttgaaatgcttgtttctcattttgtttgtgcaagagatattttcttttggacgacttcatatgcttgaacattgttc

SEQ ID NO: 142 - DNA, 350 bp, Nicotiana tabacum
  plasmid g7-1-4
gctggtgatcaaggctttggagatatcaaagataaaattatgataatgaatttcaagaattccaatggccagaatttgtcaaagaattcagatttatggaatttggaagagtgaagaagagggaaagattggaaaacatctttattgatcacttctgcaaacaacaacgagtagaggctgatttagaatttaaagtttaagagtttttataaatttagagttaaatatttgtatatatttaatgaattgtttaatatatatacaatatcgtcaataggttattatacaaatgataagtttttgtagggagtgtaaaggaaaaagttttgaaaaagaggaggatttgtttc

SEQ ID NO: 143 - DNA, 481 bp, Nicotiana tabacum
  plasmid g9-2-2 ; homology with P glycoprotein-MDRP (ATP binding cassette protein)
gcgagggccatagtgaaaaatccgaaaatcctactattggatgaggcgacgagcgcattggatgcagaatcagagagattagttcaagatgcacttgaccgggtgatggtaaatcgtacaaccgtggtggtagcacatagattatcaaccattaaaggagcagatgtaattgctgtagtcaaaaatggagtgatcgtggagaaagggaagcatgagactcttatcaacatcaaagatggtttttatgcctctttggtggccctccacacgcgtgcttcttagttctacttttttttcattaagtaaattgtattcattttaatttcgttatctttttgacttttgctgaagaagagtttctttaatagtgtactgcaactcatataaagcatagtatagtagcattcttcaattaccaaatgagagaagcaagtaaacttgcctccccgacttgacttgatgtgttctggttattaagttc

SEQ ID NO: 144 - DNA, 480 bp, Nicotiana tabacum
  plasmid g9-2-6
agcaggactagtcaagttgcatcttcacattagaaatgcttgtatatatgtgtatcagcctatcaggtagatgtgctagaaagtttttaggagcagatacaaccctggaaacctgtacagcttcttacgtcccttttatacctgtactataagtaggtaggtggtggcctgaaatcccataagccaaaaaaaatatacaagtaagcttcaccatgctccattacttagaaactgtacagcttgtgatttaccaaatatgtctacattagtcctaatatttccttagatatacgtagcctaagtattaagtcaaacctgagtttttcgaagggaaactttttgtagcaattcccttgatgttgttgactaacttctcagcagttgcaagtgaatttcatttattgtttgctattttcctgctgcgtatgttctctcttaaaattgtaaaatgtttctgtttgtttcacaccagcttcatcc

SEQ ID NO: 145 - DNA, 447 bp, Nicotiana tabacum
  plasmid g9-3-17
tggggacagcaaaacctccttggttgtgccagtgcaaagattcaagtgtaacattaaacagggaacatgctcagggaaagctgaagatcgtagatgtctgaagttagttttcccacgttttcactattttagcagagatccagaaggaagaggaggaaaagcgttctaccttaagcagctagtcgtgttgtatcgtgcatatttcatttctggtttggttttagatacttctatgtacataaactatcaaggtatttatatatgttcatattttggctttagctttcatttcatatgcacattcggctgtgggtctcctctgtaaaataatgagttctatatcattataagcattaagcttctcttgtaattgtatcagtaatattaatctcttcatttcattagttccatgactcaaccatcagcagttaataaagagtttgtttc

SEQ ID NO: 146 - DNA, 450 bp, Nicotiana tabacum
  plasmid g9-3-4
cagtgatagcaaatcaagtaattttgaaggggcagctgatggttctcacaatgttggtcagagatacagagagaggggtcagggtcagtcaaagcgtggaggtgggaatttccatggtaggcaaggtggctctggccgaataaatgccaattatgattgattgatgaggaggctaaaatgtggatttaggtctttttagtttgtgatggatagcaaacttaccggataatctttgcttagtctgcatgtctggtggtgcagtcttaggtggtagcttttgacgtggtaaaagagaatttgttggccaatgtcacacgggtgagctggactacagccgggttttgccacatggttttgggaaaaattattgtgtttggtgcaacagtaagtgcggcattatgagaactgtaattaatttgaagaacattaaaatagttgcccattttctcc

SEQ ID NO: 147 - DNA, 335 bp, Nicotiana tabacum
  plasmid g9-5-5
ggaaacacagaggcagagatgatggtgacgaggagattgacagatacttgggagttaagaacgggaaactatcagggaagctatcaaagaagccaaagagaaaatgaggaatatataattaagctattttagtccaattttgacttaattgaggaatattataattaagctatgttagttcaattttgaacttaattagttctttcattattccttgttgggctgtaatttgacatttctgcaattctgctgggatggttttgatcttagggactctattattttcattttcttgtgaagatccttgcctcctaatcctaatatatacgtgcacc

SEQ ID NO: 148 - DNA, 245 bp, Nicotiana tabacum
  plasmid g9-6-1 ; homology with LOX lipoxygenase
gtgaaagtggacttactggaaaaggaattcccaatagtgtctcaatttgaggttctacagcacgaatagctgatatatagcttttgcagtcctcgtcaacctgcagaaatcatccgcaacttaagcaggagtggcaacagatgtgtgtagatctatttttatgtcaatatttgtttagccaaattccattattgttagtgtgtgtttttacaataaaatcaatgagcaaatcccctcattttccc

SEQ ID NO: 149 - DNA, 353 bp, Nicotiana tabacum
  plasmid t12-1-7
gcattgcggtgcctatccaaagatcctcggtttagatcaagcatgagtgacattgttaaagaactagagcaactttatcaacaatctaaagatgcaggtaatactcgcagccacggtaacaaccggcctagaccacgtagncgaagtgctggtgatgttggtaataaacatacttcagttgcttatccaagaccgtctgcttctcccctttatgctaaataattcaataaatgatatgatgccttttcatgttttgcctttatgtttttcaagctgaagaacctgcacatttgcagaatcagctgattgtacagttgttttggttaatgtattggatgtgtttgtaaccttga

SEQ ID NO: 150 - DNA, 351 bp, Nicotiana tabacum
  plasmid t12-2-1 ;  homology with chitinase class 4
gtaatataatcgtatattctttttaaaatanaatcatgtatagtggagtctnatgcaattctcanaacatatatatgtcgncctcactaccgggggagcaactaatantgaatatctnnggttatnctttgattcaactnctggnnatnacttacgtcctaacatgtnagattatccccagtctccagacccagtngttgacganactcagtataatactcagcccttcnggcaacagtctgaaggtgganctccgncacatncnatctggccattaatggctcaaatggttgggccaagaccttgggnanaagntgatgaaagaatgggngnttggtncgnncgatanca

SEQ ID NO: 151 - DNA, 352 bp, Nicotiana tabacum
  plasmid t12-2-18
gaatagttgaacttatttttcaaatggcanaaatggactgacttaacttctgtacatnagctataaagatgataatcagagtgcctnctgcatntcatcctcttcttggaantgcaagaactggaagcccttcattgatgtggagtgtaaacgtggtnctataagttanttctttcgtgtcgtctgatagtttgaacctganganatgaagaagagctantggnnaagatctncatgngtcaataaangagatcttngcctaaacanattcgnggacnagcgtgaaatgntagggaatgtgaatggtaacgctggnctggaagaaganccnntccngncaagncaanctttc

SEQ ID NO: 152 - DNA, 424 bp, Nicotiana tabacum
  plasmid t18-2-5 ; homology with basic PRB-1b [I]
gttcgatgcaacaatgggtggtattttataacatgcaattatgatccacctggtaattggagaggacaacgtcctacggtgatcttgaagagcaacatccctttgattccaagttggaacttccaactgatgtctagtaataacggtttacgtgatcaaataatgaataaaagctttgtcatgtgttaaggaaaattaaataaataccagtactatgctatgtgatgttatcttcttacccagtggataataatccaatggtgtagcaaggggtggatttactgttatctacttgttttacatttgtttttggtggtattatggaggtgtgtatatgtatgtgttttgatgaataaacaaagtgaacaaggtgatgagtcaacagcgatgtaaatttgttctttgattaatataattacttact

SEQ ID NO: 153 - DNA, 277 bp, Nicotiana tabacum
  plasmid t18-3-2
ttcaaagttttcgttgccctaccaaccaccggtggatgtngctcctccngcccacaagtnaacctgatatcttnttgttttcctntagtactagaaaaatataangtagtattagttttncattctttcaatgtgtgcagttacatccctatcttttgggaggatacatcatcctcgncatcattggacttgaagtaccaccttaatcngtaaccacaatttttnaactttaaataatatcaaatttataatgacaaatatgttncttctccacttc

SEQ ID NO: 154 - DNA, 366 bp, Nicotiana tabacum
  plasmid t18-3-6 ; homology with chloroplast RNA binding protein
gtactatatgatggtgagactgggagatctcgtggctatggtntttgtgagctatgagaatagagaacaattggagaatgcccttcaaaatcttaatggagtggaactggatggaagggcaatgcgcattagcttagcacaagggaagaaacaataagatggacaagattcttgtatattagttgtaaaagttgaaaatttaccatcaatagaagaacaatgttttattcatggattaagatggctaaaggcttttaactaggacaaagggagatgtaccatttgaattacatcttccataggttgagctttctatctttgtttctttactgcctttcataatttagagatatcattgttcctttc

SEQ ID NO: 155 - DNA, 282 bp, Nicotiana tabacum
  plasmid t18-4-18 ; homology with AGP-b (ADP-G pyrophosphorylase, small sub.)
gtaatcaccggtttttattttaaacgaataatttttacagtacctantctnctcttgtaggggtaatgagaantatctagctacataaaagtnggatgtgcgctanatttctacaggnaaagcaaaatnaaagtagaanatttctaccgcatggctgttnacccaagatttgggaggacaaccaagtnccaangcctnccttcanatgataatgccactgggaatcaatgngtccttgatnacngtganaatcccnctctagannaagtatccatctgtttc

SEQ ID NO: 156 - DNA, 376 bp, Nicotiana tabacum
  plasmid t2-1-1 ; homology with ubiquitin conjugating enzyme
accagaaattgctcacatgtacaagaccgacaggtccaaatacgagaccactgctcgtagctggactcagaaatatgctatgggataatggcaaaggcgtcaccaggcatgtctgagactttgtaacagcaatgtcttattgtgctggtggtgaatgaataaattcggcgaaagaacttagtttacttcttaatctcccttaaagtgggttgtcaagagacatgtcttttcaatttgtgaatatctatttgatgactattagtaagggagaaacttcatgtaattttactttgtttgccagtttacctgagcctttctctagtttttccaatttgcctggcttgtttggttctgcgttcaaagttggtattgattc

SEQ ID NO: 157 - DNA, 364 bp, Nicotiana tabacum
  plasmid t2-1-3 ; chloroplast genome [I] homology
ggnnnncaatngnnatcgnacnagnnnncngnannannantccaaagctntcnaatnttctccattacttgtgtggataagcccnatatnatagagtatataacttcgatcatagggatcaatttctagtcgcatagcttcataataattctgcaaagcttccgcgctaatttccttcggatctgagccgacatcccatctctgtaataggtaaatgcctctttttctcctgaagttgtcggaattactcgtaatangatattggctacaattgaaaaggtcttatcaataaaatttccatttatccgtgatctaggcataggtagcaatccattctagaattcttctcattacctctcatggg

SEQ ID NO: 158 - DNA, 184 bp, Nicotiana tabacum
  plasmid t2-6-3
gagatcagtatacatgaaatggtatatacgaggacatagtttcctttagggaaatgtcaataggttagagaagaatggttaaaccgccggcccgacggtttaattaggttattatataattaggtttatcttttgacttgtatgttattagctagtaataatatacttattcaattttgtgccc

SEQ ID NO: 159 - DNA, 534 bp, Nicotiana tabacum
  plasmid t7-1-12 ; homology with SNF-1like kinase, calcineurin B-like calcium sensors interacting protein in Arabidopsis
ccagattaagcttcagggggagaagaccgggcgcaaaggtcatttatccgttgcaaccgagatttacgaggtggcaccttcactatacatggttgcttcgcaaggctggaggagataccttggaatttcacaagttttacaagaacctgtctaccggattgaaagacattgtttggcaactgggggaaggaggagaggaagtaaaagatggtcttgtcgcagcttgattttggagtgtgaagtcagtggtttgccaatgtgaataactctgcaaacagtgtgctagatattagataatgctgtgctgtaaaaagaactttttataatcagttgatgtcaaacagagtgtttaagcatcaacgagtttataatacattgttttatgtacgattaaggcacgtaaacttagaaaaattaagactggttttacattgccattgttgtcttatttggtgacaagatattacggatcaataccccccccaaaatatgtgcttttattgaactggaagtggtaacaaagtgtgttatata

SEQ ID NO: 160 - DNA, 398 bp, Nicotiana tabacum
  plasmid t7-2-4 ; homology with a multi-functional protein -beta oxidation
cctcagaaacgcaatggaggtgtcatgttttggggntgatacaattggatctgaatacatatactcaaagctaaaaacttggcatgaggcctatggtgatttttataagccatcaacatttttggagcagagagctgcaaaaggattgcccttgggaggatcgtgttgagctgcatatcatatgatcatatccttgcagaagaagcagtaattcaagcatgctgaacttgtgntcggaaataaggcgggnaagtttgttaattacaattagttagnagttccattaattataataatttcctattttttcccctcaagttatttgatggtagttgtaactttggctctacaaantagtgtaatcgtccgagaaagagaatgaaatgtccaaacgcttc

SEQ ID NO: 161 - DNA, 398 bp, Nicotiana tabacum
  plasmid t7-4-7 ; homology with GST (bronze-2 protein homologue)
atggggttgctagatatcatgatcattattacactaggggcatacaaagcacaagagctagtgtttggtgtgaaaatattggatgcagagaagacacccctcttatactcatggttgactagtttaattgagctgcctatagttaaggaaatcactcccccttatgacaaggtgctttcatttcttcatcttctcaaagacatcgtcttcaaagctccggccaattgaccttttttgtgtttatgtccatctctgtctcttttgtctactccactcattaattgtactcaatgtcttctcctctgtattgtataatataataaggcttatggccatttggattccaaaggctacttatattttgagtgtgtgttttatacaacagaaagttatcatcc

SEQ ID NO: 162 - DNA, 397 bp, Nicotiana tabacum
  plasmid t7-4-8
ccatgagaatgacgaaagcaaggcagaaaagaaaggagaacatgataagaagaatttgatgaagaaggttgctgggaaaatagggaaaaaattattgcatagtcatcctaagaagcagcatgaggaaggctatgaaggagaagaggaggaagaaggagaagaaggagaagaagtagaaggagaagaagtagaagtagaagaagcggnagaaggtggttttgaatttgaactcnactttgatttttgattaagctttatgtatcactccagctgtgtacgttggtatttctccttattggtttaaaaanacataagtatgtttcgaggatatctctgaataggtggcttggnatttgtaacctgtggtaccatatatatgagcgtcttctagtttttt

SEQ ID NO: 163 - DNA, 304 bp, Nicotiana tabacum
  plasmid t7-5-4
acgaatgtgtttagtactcggggcaactccaagtcttgagatccaagtgttgcagcctctttagcctttaaaaggtggatgctgccattttaacctggttttagtttggatgaaatttgaattcaaagcttttgtttgtagcttaggttcctgtattagttttcagttgaaatagttgtgtactctttcatctttgagcaatgaaataaaagtcctcaaatctgcttcttttagaactaaaaaagatctcttatattttcccctgtaaaatcttgcaattgattatcaaccgtcctctcttatt

SEQ ID NO: 164 - DNA, 307 bp, Nicotiana tabacum
  plasmid t7-5-5 ; Arabidopsis genomic homology
gagctgataaatggaaaagcagcagtaattggtttcctattgctgttggattttgaactcttgaccggtaaaggtcttctcaaaggaacagggttcttggatttcatttactcagtttcagatgctttcaaataaaaccattccgctatatacttactccccctccctctttttccccttttcctatttttctgacaaatttgcatttgtttaaataaacaaaaacaaagaatgttgatctttttatatgttgtccaattatatggattagtgaattatagaccattgaattccagctgaagaatgt

SEQ ID NO: 165 - DNA, 192 bp, Nicotiana tabacum
  plasmid t7-6-4
aacaataattggctataacattcaaaaatatttgaaacaagcgatgccgttacgtagaggttttacggtaaaagtagaagctggtataagccatcaatggaaaaactggataattcgatcttatataaatttcctaatgtattgagactaatatatacagtcggattttaaggttttggccgaccggattac

SEQ ID NO: 166 - DNA, 232 bp, Nicotiana tabacum
  plasmid a1-1-17
agagaaagatctgtacgtaattgccaaaaacgatgagtgtttggatgtcatgctttattttggtgtttatnggtgtctcccttttgtatttgaagttttcccagaaaattagcaaagaataagcttcaaactggttttacattttnggttcaaaatgtcanatcaaanaatctgtnatgctattggtgttgtatgtaataattagatcccattttcttcctctttcctttat

SEQ ID NO: 167 - DNA, 489 bp, Nicotiana tabacum
  plasmid t7-1-14
ccctcagaacgcaagtagcaacagtttcttcaattgctattgcctatctctgaactcgaattcattacttgtaagatctgctaataatcactatgtttttctgcagtggaggtgtcatgttttgggctgatacaattggatctgaatacatatactcaaagctaaaaacttggcatgaggcctatggtgatttctataagccatcaacatttttggagcagagagctgcaaaaggattgcccttgggaggatcgtgttgagctgcatatcatatgatcatatccttgcagaagaagcagtaattcaagcatgctgaacttgtgctcggaaataaggcgggaaagtttgttaattacaattagttagaagttccattaattataataatttcctattttttcccctcaagttatttgatggtagttgtaactttggctctacaaactagtgtaatcgtccgagaaagagaatgaaatgtccaaacgcttc

SEQ ID NO: 168 - DNA, 877 bp, Nicotiana tabacum
  CDS (31)..(588)
aacttcctctcttaaagttcatttactttgatggagaattatcaacatattcttccaaattactcttcttcatcgtctgatcagttgtcagtaatgaatatgatgaacaacaattctcaagcaaaaacaactgaattaacccaagacaataagaaatcgagcgggtttttggggctaatggcaagcatggaagctcctagctccagtgttgttactgatcacccaaatagcattccgtataaccctaatgatcagaacgaggtgagatcgggtaagaagaataaagttgagaagaagattaaaaaaccgagatatgcttttcaaacaaggagtcaagtggatattttggatgatggttatagatggaggaaatacggacagaaggctgtcaagaacaacagattcccaagaagctactaccgatgcacgcatcaaggatgtaacgtgaagaaacaagtacaaaggctgtcaaaggatgaaggagtagtagtaactacttatgaaggcatgcattcacatcccattgagaagtccacagataactttgagcacattttgactcagatgcaaatctatgcttccttttgaaacgtccatcacttcaatgcctaaggcatgacactcaattagtcacttgtaaaatagtactacagtatattgtgtacatgcgttttgaacctagatgctatattttgaaataaaacgcaacttcattagggaatttaatttgatcattgtacaactaaaagtaatgttgctatttttttgtttttatcactttgtttttgccggagccatgctcttcattttaactcttttcttttagaattaacaaataatttcatgttggagaaagatacgtgccaaaaaaaaaaaa

SEQ ID NO: 169 - protein, 185 aa, Nicotiana tabacum
MENYQHILPNYSSSSSDQLSVMNMMNNNSQAKTTELTQDNKKSSGFLGLMASMEAPSSSVVTDHPNSIPYNPNDQNEVRSGKKNKVEKKIKKPRYAFQTRSQVDILDDGYRWRKYGQKAVKNNRFPRSYYRCTHQGCNVKKQVQRLSKDEGVVVTTYEGMHSHPIEKSTDNFEHILTQMQIYASF

SEQ ID NO: 170 - DNA, 21 bp, Artificial Sequence
  Description of Artificial Sequence primer EVVRA 26
cgcgcagactctcgagggccc

SEQ ID NO: 171 - DNA, 30 bp, Artificial Sequence
  Description of Artificial Sequence primer EVVRA 28
ctcagatctagaagttcatttactttgatg

SEQ ID NO: 172 - DNA, 30 bp, Artificial Sequence
  Description of Artificial Sequence primer EVVRA 29
attgaagatctagacgtttcaactcgaggc

SEQ ID NO: 173 - DNA, 25 bp, Artificial Sequence
  Description of Artificial Sequence primer EVVRA 30
ccctcgagccaccgtactcgtcaat

(19) United States
(12) Patent Application Publication
(10) Pub. No.: US 2004/0209273 A1
(43) Pub. Date: Oct. 21, 2004

(54) Plant stress-regulated genes

(76) Inventors: Eva Vranova, Gent (BE); Dirk Inze, Moorsel-Aalst (BE); Frank Van Breusegem, Brakel (BE); Wim Van Camp, Sint-Denijs-Westrem (BE)

Correspondence Address: TRASK BRITT, P.O. BOX 2550, SALT LAKE CITY, UT, 84110, US

(21) Appl. No.: 10/647,625

(22) Filed: Aug. 25, 2003

Related U.S. Application Data
(63) Continuation of application No. PCT/EP02/01993, filed on Feb. 22, 2002.

(30) Foreign Application Priority Data
Feb. 23, 2001 (EP) 01200659 9

(51) Int. Cl. C12Q 1/68; C07H 21/04; C12N 1/08

(52) U.S. Cl. 435/6; 435/270; 536/23.6

(57) ABSTRACT

The present invention relates to a method to isolate plant genes or gene fragments that are regulated by stress, preferably oxidative stress. The method includes isolation of plant material, adaptation of the plant material to stress, differential expression of genes or gene fragments in adapted and nonadapted plant material, and isolation of the differentially expressed genes or gene fragments. The invention further relates to the genes or gene fragments that can be obtained by this method and to the use of these genes or gene fragments to modulate plant stress tolerance.

### CROSS-REFERENCE TO RELATED APPLICATIONS

This application is a continuation of international application number PCT/EP02/01993, filed Feb. 22, 2002, designating the United States of America, published in English Feb. 13, 2003, corresponding to international publication number WO 03/012096 A2, the contents of which are incorporated herein by this reference in its entirety.

### TECHNICAL FIELD

The present invention relates to a method to isolate plant genes or gene fragments that are regulated by stress, preferably oxidative stress. The method comprises isolation of plant material, adaptation of the plant material to stress, differential expression of genes or gene fragments in adapted and nonadapted plant material, and isolation of the differentially expressed genes or gene fragments. The invention further relates to the genes or gene fragments that can be obtained by this method and to the use of these genes or gene fragments to modulate plant stress tolerance.

### BACKGROUND

Plant molecular responses to environmental stresses are generally very complex and often result in alteration of gene and protein expression, as well as in changes in metabolic profiles (Sandermann et al., 1998; Jansen et al., 1998; Somssich and Hahlbrock, 1998; Bartels et al., 1996). At least some of those stress responses are required for enhanced stress tolerance as the moderate doses of many stresses increase plant resistance to deleterious stress conditions. For example, raising the temperatures slowly to high, nonlethal temperatures allows plants to tolerate temperatures that are normally lethal, a phenomenon referred to as acclimation (Vierling, 1991). Similarly, exposing maize plants to 14° C. acclimates them to lower temperatures that would normally cause chilling injuries (Prasad et al., 1994). Also, pathogen infection often leads to resistance against subsequent challenges by the same or even unrelated pathogens (reviewed in Sticher et al., 1997). This phenomenon of induced stress tolerance is not restricted to the same kind of stress and cross-tolerance induced by different kinds of stresses have been reported (Orvar et al., 1997; Örzech and Burke, 1988; Keller and Steffen, 1995; Cloutier and Andrews, 1984).

Much of the damage due to environmental constrains has been attributed to the excess production of active oxygen species (AOS), so-called oxidative stress (reviewed in Inz6 and Van Montagu, 1995). Plant cells acclimated to heat and cold, as well as plants expressing systemic acquired resistance to pathogens, also show enhanced capacity to tolerate oxidative stress (Banzet et al., 1998; Seppanen et al., 1998; Strobel and Kuc, 1995). This suggests that induced tolerance to oxidative stress is part of the adaptation mechanism to environmental stresses and likely contributes to the observed phenomenon of cross-tolerance. However, little is known in plants about molecular mechanisms underlying induced tolerance to oxidative stress.

In contrast, adaptive responses to various oxidants have been extensively studied in bacteria and yeast. In both E. coli and S. cerevisiae, adaptation to oxidative stress is an active process requiring de novo protein synthesis (Davies et al., 1995; Storz et al., 1990). At least 80 proteins are induced by adaptive amounts of oxidants in E. coli; 40 of them belong to H₂O₂ stimulon and 40 to O₂ [symbol] stimulon. Among the induced enzymes are antioxidant enzymes, DNA repair enzymes, heat shock proteins, and glucose-6-phosphate dehydrogenase implicated in energy homeostasis (reviewed in Demple, 1991).

Yeast, similarly to bacteria, possess at least two distinct but overlapping adaptive stress responses to oxidants: one induced by H₂O₂ and the other by O₂ [symbol] generating compounds (Jamieson, 1992). The H₂O₂ stimulon has been analyzed by comparative two-dimensional gel analysis of total cell proteins isolated after treatment with low doses of H₂O₂ (Godon et al., 1998). Such a treatment resulted in synthesis of at least 115 proteins and repression of 52 proteins. 70% of those proteins have been identified and classified into cellular processes such as antioxidant defenses, heat shock responses and chaperone activities, protein turnover, sulphur, amino acids, purine, and carbohydrate metabolism. Notably, carbohydrate metabolism was redirected to the regeneration of NADPH, which provides reducing power necessary for the detoxification of active oxygen species.

In plants, tolerance to oxidative stress has been previously associated with enhanced activity of antioxidant enzymes and levels of antioxidant metabolites (reviewed in Inze and Van Montagu, 1995). In addition, Banzet et al. (1998) have demonstrated that other stress proteins are likely implicated in acquisition of oxidative stress tolerance by plant cells, similarly as in lower organisms. Expression of small heat shock proteins correlated with adaptation of tomato cells to oxidative stress and, additionally, heat shock pretreatment was able to enhance resistance of those cells to oxidative stress. However, no comparative genome-wide characterization of induced adaptive responses to oxidative stress has been undertaken in plants.

### SUMMARY OF THE INVENTION

A genomic approach was used to study the adaptive responses to oxidative stress in leaf tissue of Nicotiana tabacum. The redox-cycling compound, methyl viologen (MV; paraquat), was used to induce an adaptive response to oxidative stress, as AOS signaling may be important during the defense against both biotic and abiotic stresses in plants (Levine et al., 1994; Prasad et al., 1994; Banzet et al., 1998; Chamnongpol et al., 1998; Alvarez et al., 1998; Karpinski, 1999). Surprisingly, we found that adaptive amounts of MV enhance the tolerance of tobacco leaf tissues to oxidative stress imposed by toxic levels of the same oxidant. Moreover, adaptation to oxidative stress is associated with induction/repression of approximately 170 genes, and partial characterization of induced genes shows that they are implicated in distinct cellular processes. Several of these defense responses induced by adaptive amounts of oxidants have so far never been associated with the antioxidant response.

It is a first aspect of the invention to provide a method to isolate stress-regulated genes or gene fragments, the method comprising:

(a) isolating plant material;

(b) inducing stress adaptation in plant material;

(c) checking differential expression between stress-adapted and nonadapted plant material; and

(d) isolating differentially expressed genes or gene fragments.

Plant material can be any plant material, such as parts of, or complete, roots, stems or leaves. Plant material may include more than one plant tissue, up to a complete plant. Preferably, the plant is a tobacco plant. Even more preferable, the plant material is leaf material.

Induction of stress adaptation is preferentially carried out by applying sublethal stress to the plant material. Stress can be any biotic or abiotic stress, such as fungal or bacterial infection, heat or cold treatment, or oxidative stress. Preferably, the stress is oxidative stress. More preferably, the oxidative stress is applied by putting the plant material in a solution comprising an adequate amount of methyl viologen (methyl viologen pretreatment). Alternatively, the sublethal stress phase may be followed by a period of stronger stress. The stronger stress may even result in significant cell damage when applied to unadapted plant material.

Differential expression includes induction as well as repression. Checking differential expression can be done with all the differential expression or differential display techniques known to the person skilled in the art, such as, but not limited too, messenger substraction, filter hybridization or micro-array techniques.

Isolation of the differentially expressed genes may be direct or indirect, i.e., by direct isolation of the differentially expressed nucleic acid such as mRNA or cDNA, or by isolation of the genes from a library, on the basis of the results identifying the gene, such as filter hybridization or micro-array. Preferably, the differentially expressed genes or gene fragments are isolated using PCR-based techniques.

A further aspect of the invention is a gene, or gene fragment, obtained by the method according to the invention. A preferred embodiment is a gene or gene fragment, comprising a sequence selected from any of the sequences from SEQ ID NO: 1 to SEQ ID NO: 167.

Clone names of these sequences, their expression pattern and an indication of the function by homology search is summarized in Table 1.

In one embodiment, a gene encoding a protein comprising, or preferably essentially consisting of, or more preferably consisting of, SEQ ID NO: 169. Preferably, the gene comprises SEQ ID NO: 168. More preferably, the gene is essentially consisting of SEQ ID NO: 168 and, even more preferably, the gene is consisting of SEQ ID NO: 168.

Still another aspect of the invention is the use of a gene or a gene fragment according to the invention, or a gene that is at least 60% identical, preferably 80% identical, and more preferably 90% identical to the gene or gene fragment according to the invention, or a gene fragment from a gene that is at least 60% identical, preferably 80% identical, or more preferably 90% identical to the gene or gene fragment according to the invention to modulate plant stress tolerance. A preferred embodiment is the use of a gene or gene fragment comprising SEQ ID NO: 168, preferably essentially consisting of SEQ ID NO: 168, or more preferably consisting of SEQ ID NO: 168. Preferably, the stress is oxidative stress. Preferably, the plant is tobacco.

A special embodiment is the use of a gene fragment according to the invention, whereby the gene fragment is a promoter. Although the gene fragments isolated by the differential expression procedure may be coding sequences that do not comprise the promoter of the gene, a person skilled in the art can isolate the promoter of a gene when the coding sequence is known. As a nonlimiting example, the coding sequence can be used as a probe against a genomic library, whereby the positive scoring clones are subcloned, and the positive subclone is sequenced. On the base of the sequence, the promoter part and the coding part, including the intron-exon boundaries, can be predicted using computer software, such as Genemark, Genscan or Grail. Alternatively, the full-length messenger RNA can be isolated and, on the base of its sequence, the start of transcription can be defined and the promoter can be localized.

Another aspect of the invention is a vector comprising a gene or a gene fragment according to the invention. The vector may be any vector suitable for eukaryotic cells, as is known to the person skilled in the art, and includes, but is not limited to, self-replicating vectors, integrative vectors and virus-based vectors. Preferably, the vector is a plant transformation vector and the eukaryotic cell is a plant cell.

Still another aspect of the invention is a method to modulate stress tolerance in a plant cell or plant comprising the introduction of the vector according to the invention in the plant cell or plant. Introduction of the vector in the plant cell or plant can be realized by any suitable technique known to the person skilled in the art and includes, but is not limited to, transformation techniques such as electroporation, particle bombardment or Agrobacterium-mediated transformation, floral dip transformation or sexual techniques such as crossing.

A further aspect of the invention is a plant cell or plant, comprising a vector according to the invention. Preferably, the plant cell or plant is a tobacco plant cell or plant.

### BRIEF DESCRIPTION OF THE DRAWINGS

FIG. 1. Effect of different concentrations of methyl viologen on leaf disc damage. Three leaf discs were floated on solution with assigned methyl viologen concentrations for indicated time periods. Ion leakage was measured as conductivity of the medium at indicated time intervals. The experiment was done in duplicate and the presented value is the average of both measurements. The conductivity of the solution was subtracted from the measured values.

FIG. 2. Effect of MV pretreatment on leaf disc tolerance to 1 μM methyl viologen. Leaf discs that were pretreated for 17 hours with water (grey bars) or 0.1 μM methyl viologen (black bars) were exposed to 1 μM solution of methyl viologen. Ion leakage was measured as conductivity of the medium in the course of the treatment at regular intervals. The conductivity of the solution was subtracted from measured values. Presented values are average values of nine independent experiments.

FIG. 3. Expression of GPx and SodCc during the treatment with 1 μM methyl viologen. Leaf discs pretreated with water (0) or 0.1 μM MV (0.1) for 17 hours were exposed to 1 μM methyl viologen and expression of a glutathione peroxidase gene (GPx) and a gene encoding cytosolic CuZnSOD (SODCc) was analyzed. Total RNA (5 μg) was extracted from six leaf discs sampled in two independent experiments at indicated times and subjected to Northern analysis. The same membrane was used for hybridization with both genes. Hybridization of the constitutive actin gene was used as a loading control (bottom panel).

FIG. 4. Expression of genes isolated by differential display during the pretreatment with 0.1 μM methyl viologen and the treatment with 1 μM methyl viologen. Total RNA was extracted from nine leaf discs sampled at indicated times before (c) and during the pretreatment with 0.1 μM MV (0.1) or water (0), and after exposure of pretreated samples to 1 μM MV. Blots with 15 μg total RNA each were prepared in quadruplicate and checked for equal loading by methylene blue staining. Each membrane was reused several times.

FIGS. 5A and 5B. Resistance to MV of A. thaliana transformed with WRKY11 fused to the VP16 activation domain, under control of the 35S promoter. (A) The control plate without MV; (B) the test plate with 2 μM MV. WV9 and Wv4 are transformed lines; C24 is an untransformed control.

### DETAILED DESCRIPTION OF THE INVENTION

While this invention is described in certain embodiments and by way of certain examples, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

Definitions

As used herein “Plant material” can be any plant tissue such as root, stem or leaf. It may be a part of the plant, such as a disc excised from the leaf, up to the intact plant.

As used herein “Adaptation” means the application of a stress to the plant for a certain time, whereby the time and/or the level of stress are controlled in such a way that the stress applied over the time used is sublethal.

As used herein “Sublethal stress” refers to stress that may result in a specific gene expression pattern but is not leading to a lethal amount of cell damage. Detrimental tissue damage can be evaluated by several methods known to the person skilled in the art, but is preferably evaluated by measuring an increase in conductivity as described in the examples. An increase in conductivity in the stress situation, compared with a nonstressed reference situation by less than a factor 5, preferably less than a factor 2, as measured after 42 hours of stress application is considered as insignificant.

The term “gene,” as used herein, refers to a polymeric form of nucleotides of any length, either ribonucleotides or deoxyribonucleotides. This term refers only to the primary structure of the molecule. The term includes double- and single-stranded DNA and RNA. It also includes known types of modifications, for example, methylation, “caps” substitution of one or more of the naturally occurring nucleotides with an analogue. It includes, but is not limited to, the coding sequence. It does include the regulatory sequences such as the promoter and terminator sequences.

As used herein “Gene fragment” may be any gene fragment of at least 40 contiguous nucleotides, preferably 60 nucleotides, more preferably 100 nucleotides, either coding or noncoding. A special case of gene fragment is the promoter of the gene.

As used herein “Modulation of stress tolerance” comprises both the increase of stress tolerance, as well as the decrease of stress tolerance, independent of the level of decrease or increase.

As used herein “% identical” is the percentage identity between two or more nucleic acid or amino acid sequences as measured by a TBLASTN search (Altschull et al., 1997).

Plant Material and Cultivation Conditions

Nicotiana tabacum cv. Petit Havana SR1 plants were grown in a controlled environment chamber (Weiss Technik, Lindenstruth, DE) under 100 μmol/m²/s light intensity (photosynthetically active radiation), 16 hours light/8 hours dark regime, relative humidity of 70% and constant temperature of 24° C. The most expanded leaves (11-12 cm long×7-8 cm wide) from five week old plants were used for experiments with methyl viologen.

Methyl Viologen Treatment

Leaf discs (1 cm in diameter) were punched with a cork-bore from the interveinal part of the leaf. Three leaf discs, each originating from different plants, were floated with the abaxial side up on 12 ml of methyl viologen solution in nanopure water or water solely in the case of control. Treatments were performed in controlled environment chambers, under the same conditions as for growth, except as otherwise indicated. Leaf discs for RNA extraction were drained on paper, rapidly frozen in liquid nitrogen and stored at −70° C. Ion leakage from the leaf discs was measured as conductivity of the solution using a conductivity meter (Consort, Turnhout, BE)

RNA Extraction and Northern Analysis

Total RNA was extracted from frozen leaf discs using TRIzol™ Reagent (Life Technologies, Paisley, UK) according to the manufacturer's instructions. RNA samples were treated prior to electrophoresis and resolved on 1% agarose gel as described by Shaul et al. (1996). The RNA was blotted on nylon membrane (Hybond-N, Amersham International plc., Buckinghamshire, UK or Qiabrane, Qiagen GmbH, Hilden, DE) by capillary blotting (Maniatis et al., 1982). RNA was fixed to the membrane by cross-linking at 150 mJ/cm². To check the quality of RNA prior to hybridization, membranes were incubated for 15 minutes in 5% acetic acid and stained for five minutes in 0.04% methylene blue in 0.5 M sodium acetate (pH 5.2), and rinsed with water. After the staining and quality check, membranes were destained in 0.1×SSC (Maniatis et al., 1982) containing 0.5% SDS (w/v). Membranes were hybridized at 65° C. in 50% formamide, 5×SSC, 0.5% SDS and 10% dextran sulphate. ³²P-labeled RNA probes corresponding to the cDNA fragments of GPx (Criqui et al., 1992), SodCc (PSOD 3-5′ fragment; Tsang et al., 1991), SodB (pSOD 2-5′ fragment; Tsang et al., 1991), Cat1 (pCat1A; Willekens et al., 1994), and N. tabacum actin (pRVA12; Aventis Crop Science, BE) were generated by the Riboprobe® System (Promega Corp., Madison, Wis., USA). RNA probes corresponding to cDNA fragments isolated by differential display and cloned into pGEM®-T vector (Promega Corp., Madison, Wis., USA) were generated according to the same protocol. Membranes were washed at 65° C. for 15 minutes each in 3×SSC (Maniatis et al., 1982), 1×SSC and 0.1×SSC (stringent washing) containing 0.5% SDS (w/v). Membranes were exposed to the Storage Phosphor Screen and scanned with the Phosphorlmager 445 SI (Molecular Dynamics Inc., Sunnyvale, Calif., USA). Membranes were reused after stripping of the probe in 0.1×SSC at 85° C. Removal of the probe was checked by autoradiography.

Differential Display

Total RNA was treated with DNaseI prior to RT-PCR according to the manufacturer's instructions (Life Technologies, Paisley, UK). Alternatively, up to 20 μg of total RNA was incubated with 5 U DNaseI, 18 U Recombinant Ribonuclease Inhibitor (Promega Corp., Madison, Wis., USA), 1 mM DTT in 80 μl of 10 mM Tris-Cl, pH 8.3, 50 mMKCl and 1.5 mM MgCl₂ for 30 minutes at 37° C. RNA was extracted with phenol/CHCl₃ (3:1), ethanol precipitated and dissolved in diethyl pyrocarbonate-treated water. mRNA differential display was performed with the RNA map™ kit (Gene Hunter Corp., Nashville, Tenn., USA), AmliTaq DNA polymerase (Perkin-Elmer, Branchburg, N.J., USA) and [³³P] dATP (0.2 μl/20 μl PCR reaction of 111 000 GBq/mmol; Isotopchim, Ganagobie-Peyruis, FR). 3.5 μl of each PCR reaction was mixed with 2 μl of loading dye and denatured at 95° C. for five minutes prior to loading onto 6% DNA sequencing gel. Gels were electrophoresed at 90 Watts constant power until the xylene dye reached the bottom and dried at 80° C. for about one hour. All the 20 decamers were used in combination with the four T₁₂MN primers provided with the kit. Bands with a differential expression pattern and larger than 200 bp were purified from the polyacrylamide gels and reamplified according to the instructions provided in the manual of the RNA map™ kit. Reamplified cDNA was ethanol precipitated and cloned into pGEM®-T vector (Promega Corp., Madison, Wis., USA). Each clone was assigned a number corresponding to the primer used, position on the gel and number of cDNA fragment within the isolated band (e.g., t 18-2-5 was amplified with primers T₁₂MT and AP18 isolated as a second from the top of the gel, and after the cloning, the fifth colony was sequenced).

DNA Sequence Analysis

Three to six cDNAs originating from a single band were sequenced by primer walking using ABI Prism® BigDye™ terminator cycle sequencing kit (PE Applied Biosystems, Foster City, Calif., USA). DNA sequence data were analyzed using the Wisconsin Package Version 9.1 (genetics Computer Group (GCG), Madison, Wis.). The nucleotide sequences of all cloned cDNAs were compared with sequences deposited in GenBank, EMBL, DDBJ, and PDB databases, and translated DNA sequences were compared with protein sequences of GenBank CDS translations, PDB, SwissProt, PIR and PRF databases using the BLAST algorithm (Altschul et al., 1997). The scoring matrix used by blastp search was BLOSUM62 (Henikoff and Henikoff, 1992). Gene homologues in the database were considered to be significant when the e-value was <10⁻³ and the high-scoring segment pair identity was at least 20% for an amino acid sequence and 50% for a nucleotide sequence.

Plasmid Construction

pWRKY11: WRKY11 cDNA was amplified from a cDNA library with primers EVVRA 28 and EVVRA 29 and cloned into pGEM-t™(Promega) PstI and NotI sites via an intermediate cloning in the pZErO™ vector (Invitrogen).

pWRKY-pGSJ780A: The BglII-digested WRKY11 sequence was cloned into the BamHI site of the pGSJ780 binary vector (Bowler et al., 1991).

pWRKY-VP16-pGSJ780A: The VP16 activation domain was amplified from pTETVP16 by primers EVVRA 26 and EVVRA30 and cloned as an XhoI fragment into the XhoI site of pWRKY11.

The WRKY-VP16 fusion was then cloned as a BglII fragment into the BamHI site of pGSJ780A.

Arabidopsis Transformation

Arabidopsis transformation was carried out by the floral dip method (Clouch and Bent, 1998). Selection of primary transgenics and progeny was based on transgene expression levels as determined by Northern blot analysis.

Stress Assessment

Eighty plants of an F₂-progeny of the transgenic line WV4 (construct pWRKY-VP16-pGSJ780A) were grown on MS+Kanamycine for two and a half weeks. Fifteen kanamycine-resistant seedlings were transferred to plates containing ½ MS, 1% sucrose and 2 μM methyl viologen (paraquat) or to plates containing ½ MS, 1% sucrose for the controls.

Wild-type Arabidopsis plants were treated in a similar way (except for selection on Kanamycine).

Performance of plants was followed and pictures were taken after approximately three weeks.

### EXAMPLE I

### Sensitivity of Tobacco to Methyl Viologen

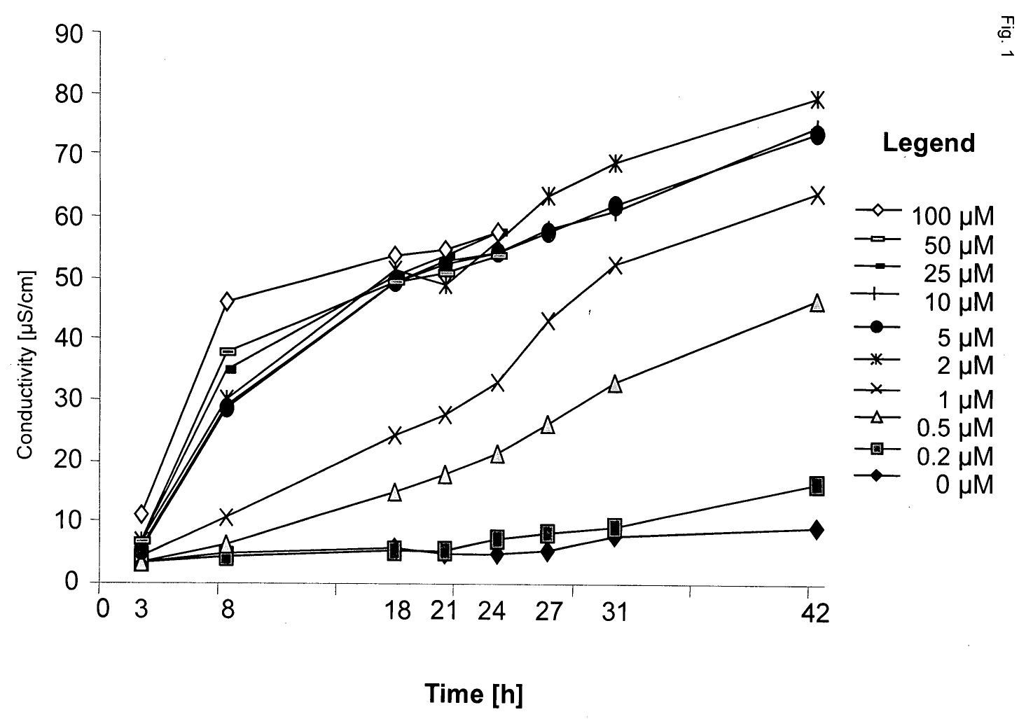

As a first step in studying adaptive responses to oxidative stress in tobacco, we wanted to establish an experimental system in which low doses of oxidant would induce adaptation to higher doses of the same compound. MV, a redox-active compound that enhances superoxide radical (O₂ [symbol] ) formation mainly in chloroplasts, was used as an oxidant. In order to determine MV concentrations suited for the induction of adaptation and for the subsequent oxidative stress treatment, sensitivity of tobacco to MV was first determined. Leaf discs were floated on solutions with different concentrations of MV and ion leakage was monitored by measuring the solute conductance. If not scavenged, superoxide generated by MV is converted through redox-reactions into other active oxygen species (AOS) such as hydroxyl radicals that interact with biological molecules and cause oxidative damage (Halliwell and Gutteridge, 1989). Peroxidation of membrane lipids results in loss of membrane integrity that is reflected by enhanced cellular ion leakage. Concentrations lower than 0.2 μM MV caused very little increase in ion leakage from the leaf discs in comparison with water-treated controls and no visible damage was seen even after 42 hours of incubation (FIG. 1). These concentrations thus seemed most suitable for inducing adaptation to MV. When leaf discs were incubated in MV solutions at concentrations ranging from 0.2-2 μM MV, leaf damage measured as solute conductance clearly correlated with the applied dose of MV. This correlation was more or less linear within this range, suggesting that these doses of MV are most suited for monitoring differences in MV sensitivity between pretreated and control samples.

### EXAMPLE II

### MV Pretreatment Induces Tolerance and Activates Expression of Antioxidant Genes

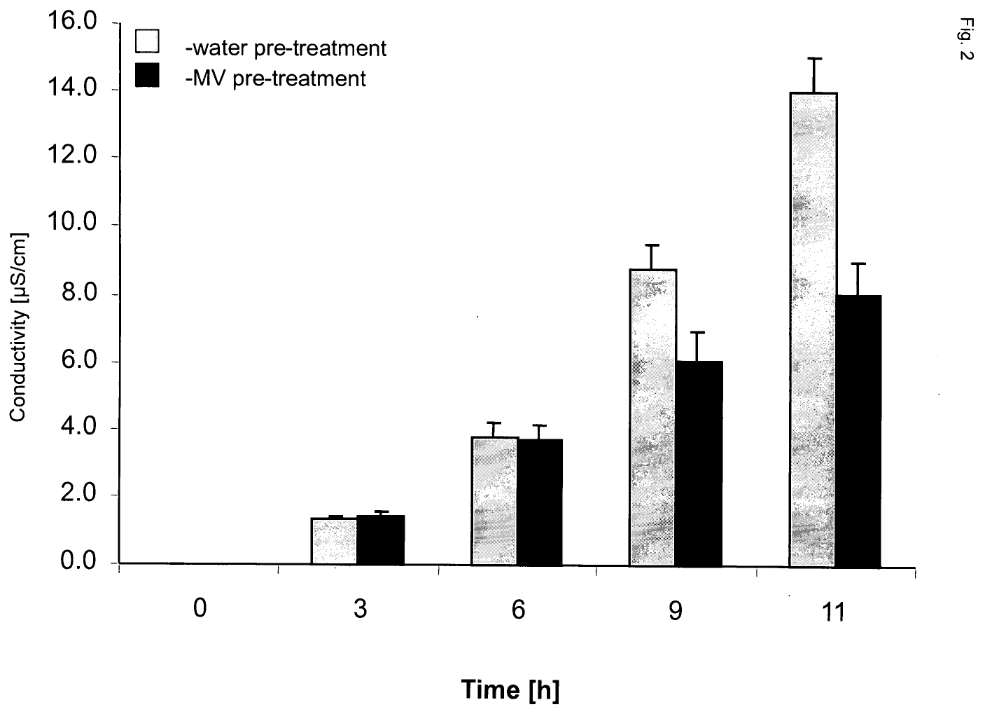

To test whether exposure to sublethal amounts of MV enhances tolerance to higher, normally toxic amounts of this compound, tobacco leaf discs were floated on solutions with less than 0.2 μM MV and subsequently transferred to solutions within the molar range of 0.2-2 μM. Increase in tolerance was assessed by measuring the solute conductance. Leaf discs pretreated with water were taken as nonadapted controls. Protection against MV was most pronounced (40% in the mean compared to water-pretreated control samples) when leaf discs were pretreated with 0.1 μM MV for 17 hours (including eight hours dark period; referred to as “pretreatment”) and subsequently treated with 1 μM MV for 11 hours (referred to as “treatment”) (FIG. 2). These parameters for the pretreatment and the treatment were then used in all further experiments, unless otherwise stated. The specific conditions required for inducing adaptation were not investigated; yet, it was noticed that both the MV concentration and duration of the pretreatment were factors that affected the level of protection.

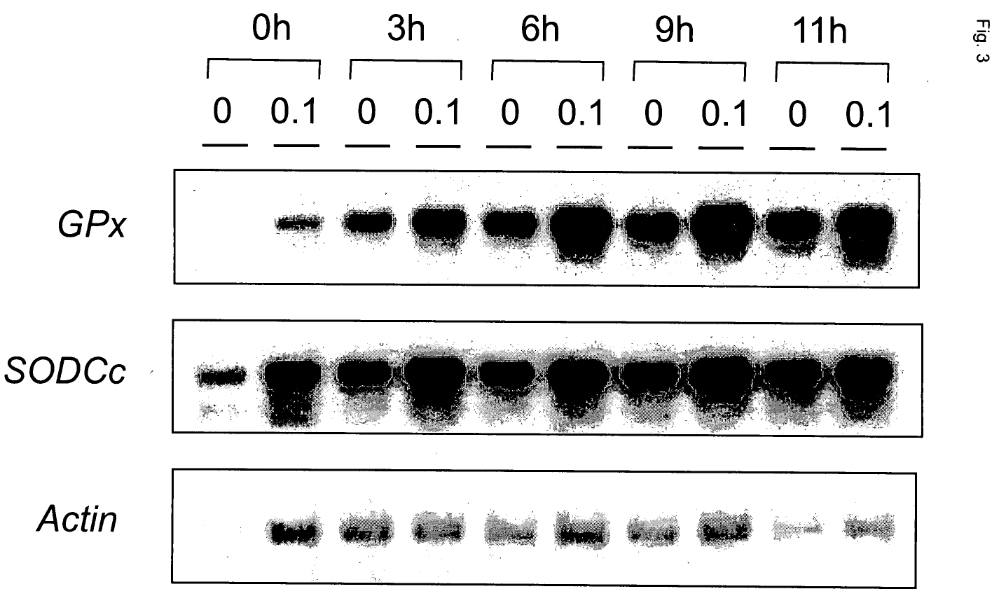

mRNA levels of several antioxidant genes were tested by Northern analysis during the pretreatment and the treatment. Both water and MV caused a rapid induction (one hour) of a glutathione peroxidase gene (Gpx) and a gene encoding cytosolic CuZnSOD (SodCc) (data not shown). Gpx and SodCc were only transiently induced in water-pretreated samples, suggesting that this induction was caused by the tissue wounding during leaf disc preparation. In contrast, pretreatment with 0.1 μM MV gave a persistent increase in Gpx and SodCc mRNA. After transfer to 1 μM MV, Gpx and SodCc were again induced in both water and MV-pretreated samples. However, the amount of both messengers remained consistently higher in MV-pretreated samples (FIG. 3). The above data indicate that induced tolerance is not just a physiological response but that it involves changes in nuclear gene expression and that GPx and cytosolic CuZnSOD are playing a role in the observed enhanced tolerance of pretreated samples. CatI and SodB genes were also induced following the pretreatment, but their transcript levels declined during the subsequent treatment with 1 μM MV and no correlation could be established between their mRNA levels and enhanced tolerance.

### EXAMPLE III

### Expression of a Large Number of Genes Implicated in Distinct Cellular Processes is Modulated by MV Pretreatment

In order to identify which genes other than those encoding antioxidant enzymes would show altered mRNA levels during oxidative stress adaptation, reference samples placed in water for 17 hours, or samples pretreated with 0.1 μM MV for 17 hours (adapted leaf discs), were compared by differential mRNA display. To increase the fidelity of the differential display results, mRNA from two independent experiments was used to prepare cDNA; and reverse transcription was performed in duplicate for each RNA sample. Amplified cDNA from two separate experiments and two independent reverse transcription reactions were displayed next to each other on the sequencing gel. Eighty primer combinations yielded 243 bands larger than 150 bp that consistently showed differential expression between adapted and nonadapted samples. Two hundred two of them were up-regulated and 41 were down-regulated. Reamplified cDNA fragments larger than 200 bp were cloned and three to six cDNAs from 60% of all bands sequenced. Sequencing data revealed that 50% of sequenced bands contained two or more cDNA species and 30% of the bands were redundant. Taking into account this redundancy and assuming that only one cDNA species per band contributed to the differential expression pattern, the total number of genes with altered expression after MV pretreatment is estimated to be 170. Expression of 16 genes was further analyzed by Northern analysis with RNA from an independent experiment. The induction of 12 genes was confirmed, while four genes remained uninduced. Three out of these four genes were isolated from bands consisting of mixed cDNAs, indicating that they were not responsible for the differential expression pattern. The fact that expression of most of the isolated genes was reconfirmed by Northern analysis is a good indication of procedure fidelity and suggests that the number of genes transcriptionally responding to MV is close to the number estimated by sequencing data.

The nucleotide sequences and translations of 167 cDNAs isolated from differentially expressed bands were compared with nonredundant databases. Only 12 cDNAs were identical or highly similar (>90% over the whole sequence) to previously isolated tobacco genes. Of the other 145 cDNAs, 36 were significantly similar to genes/proteins with known or predicted function, and 16 to genes with no assigned function. The high percentage of cDNAs (62%) for which no similarity was found in the database can, in part, be attributed to the fact that the isolated cDNAs mostly contain the 3′ untranslated region where sequence divergence is very high. The homologues of isolated cDNAs, of which the expression was tested and reconfirmed by Northern analysis, are listed in Table 2. Data shows that in addition to antioxidant genes, genes encoding chaperones (DNAJ), transporter proteins (MDR), dioxygenases (DIOX), enzymes of carbohydrate (ATPC-L), lipid (Lox2, MFP) and terpenoid metabolism (EAS, VS), regulatory proteins (WRKY11, TPK) and pathogen-related proteins (PRB1b, CBP20) are activated during MV-induced adaptation to oxidative stress in tobacco. The large number, as well as the functional diversity of genes transcriptionally responding to MV pretreatment, indicates that AOS activate a wide range of responses within the plant cells.

TABLE 1
list of stress-related genes with identification on the base of homology

| | | | homology E < 10-3 with at least 20% amino acids or 50% nucleic acids identical nonredundant |
|---|---|---|---|
| Clone number | DD+/− | N+/−/= | DNA and protein sequence databases (blastx/blastn) |
| a1-1-14.seq | + | | SEQ ID NO: 1 |
| a1-1-7.seq | + | | SEQ ID NO: 166 |
| a10-2-12.seq | + | | hypothetical protein [Arabidopsis thaliana] (gb\|AAD08932) SEQ ID NO: 2 |
| a10-4-1.seq | + | | metallothionein-like protein type 2 Nicotiana plumbaginifolia (gb\|U35225) SEQ ID NO: 3 |
| a10-4-12.seq | + | | SEQ ID NO: 4 |
| a10-4-15.seq | + | | SEQ ID NO: 5 |
| a14-1-1.seq | + | = | serine carboxypeptidase-like protein Oryza sativa (dbj\|BAA04511) SEQ ID NO: 6 |
| a14-1-3.seq | + | | SEQ ID NO: 7 |
| a14-1-4.seq | + | | SEQ ID NO: 8 |
| a18-1-5.seq | + | | EREBP-1 Matricaria chamomilla (dbj\|BAA87068) SEQ ID NO: 9 |
| a18-1-8.seq | + | | SEQ ID NO: 10 |
| a18-3-2.seq | + | | SEQ ID NO: 11 |
| a18-3-3.seq | + | | EIF-5A (eukaryotic initiation factor 5A2) Solanum tuberosum (sp\|P56333) SEQ ID NO: 12 |
| a18-4-6.seq | + | | SEQ ID NO: 13 |
| a19-3-1.seq | + | | SEQ ID NO: 14 |
| a19-3-3.seq | + | | SEQ ID NO: 15 |
| a19-3-4.seq | + | | SEQ ID NO: 16 |
| a19-3-9.seq | + | | SEQ ID NO: 17 |
| a20-1-3.seq | + | | SEQ ID NO: 18 |
| a3-2-2.seq | − | | ribosomal protein L12 (60S) Prunus armeniaca (sp\|O50003) SEQ ID NO: 19 |
| a8-1-1.seq | − | | SEQ ID NO: 20 |
| a8-1-2.seq | − | | geranyl-geranyl reductase chlP-gene Nicotiana tabacum (emb\|CAA07683) SEQ ID NO: 21 |
| a8-1-4.seq | − | | early wound inducive gene Nicotiana tabacum (dbj\|BAA95791) SEQ ID NO: 22 |
| a9-1-2.seq | + | | epoxide hydrolase Nicotiana tabacum (gb\|AAB02006) SEQ ID NO: 23 |
| a9-3-4.seq | + | | immediate-early salicylate-induced glucosyltransferase (IS10a) Nicotiana tabacum (gb\|U32643) SEQ |
| | | | ID NO: 24 |
| a9-4-1.seq | + | | SEQ ID NO: 25 |
| a9-5-9.seq | + | | SEQ ID NO: 26 |
| a9-6-11.seq | − | | SEQ ID NO: 27 |
| a9-7-1.seq | + | | SEQ ID NO: 28 |
| a9-7-10.seq | + | | lipoxygenase LOX1 Nicotiana tabacum (emb\|X84040) SEQ ID NO: 29 |
| a9-7-11.seq | + | | SEQ ID NO: 30 |
| c1-1-3.seq | + | | SEQ ID NO: 31 |
| c1-1-5.seq | + | | SEQ ID NO: 32 |
| c1-2-2.seq | + | | SEQ ID NO: 33 |
| c1-3-12.seq | − | | SEQ ID NO: 34 |
| c10-3-1.seq | − | | SEQ ID NO: 35 |
| c10-3-5.seq | − | | SEQ ID NO: 36 |
| c11-2-1.seq | + | | SEQ ID NO: 37 |
| c11-3-1.seq | + | | SEQ ID NO: 38 |
| c11-3-3.seq | + | | caffeoyl-CoA O-methyltransferase Nicotiana tabacum (emb\|Z56282) SEQ ID NO: 39 |
| c13-1-6.seq | + | | SEQ ID NO: 40 |
| c13-2-1.seq | + | | L19 ribosomal protein Nicotiana tabacum (emb\|Z31720) SEQ ID NO: 41 |
| c13-3-13.seq | + | | 23S 4.5S rRNA genes chlP-genes Nicotiana tabacum (gb\|J01446) SEQ ID NO: 42 |
| c13-3-6.seq | + | | SEQ ID NO: 43 |
| c14-1-60.seq | + | | glycolate oxidase Lycopersicon esculentum (pir\|T07032) SEQ ID NO: 44 |
| c14-2-10.seq | + | | SEQ ID NO: 45 |
| c14-2-15.seq | + | | ribosomal protein L35-like (60S) Arabidopsis thaliana (emb\|CAB85998) SEQ ID NO: 46 |
| c14-3-4.seq | + | | ribosomal protein L23a-like (60S) Arabidopsis thaliana (emb\|CAB75762) SEQ ID NO: 47 |
| c14-5-1.seq | − | | predicted protein Oryza sativa (dbj\|BAA83350) SEQ ID NO: 48 |
| c14-6-11.seq | + | | predicted protein Arabidopsis thaliana (pir\|T02387) SEQ ID NO: 49 |
| c14-7-4.seq | + | | SEQ ID NO: 50 |
| c15-1-2.seq | + | | SEQ ID NO: 51 |
| c15-1-4.seq | + | + | pathogen-and wound-inducible antifungal protein CBP20 precursor Nicotiana tabacum |
| | | | (gb\|AAB29959) SEQ ID NO: 52 |
| c15-11-2.seq | + | | SEQ ID NO: 53 |
| c15-11-4.seq | + | | SEQ ID NO: 54 |
| c15-2-8.seq | + | | hypothetical protein Arabidopsis thaliana (emb\|CAB88533) SEQ ID NO: 55 |
| c15-3-4.seq | + | | hypothetical protein Arabidopsis thaliana (gb\|AAF63779) SEQ ID NO: 56 |
| c15-6-2.seq | + | | SEQ ID NO: 57 |
| c15-6-3.seq | + | | SEQ ID NO: 58 |
| c15-7-1.seq | − | | SEQ ID NO: 59 |
| c15-8-5.seq | − | | SEQ ID NO: 60 |
| c17-3-1.seq | + | | SEQ ID NO: 61 |
| c17-3-5.seq | + | | SEQ ID NO: 62 |
| c17-5-5.seq | + | | SEQ ID NO: 63 |
| c17-5-8.seq | − | | SEQ ID NO: 64 |
| c17-6-2.seq | + | | SEQ ID NO: 65 |
| c18-1-2.seq | + | + | DNAJ protein-like Arabidopsis thaliana (emb\|CAB86070) SEQ ID NO: 66 |
| c18-2-1.seq | + | | CCT (chaperonin containing TCP-1) b subunit Oxytricha nova (gb\|AF188130) SEQ ID NO: 67 |
| c19-2-11.seq | + | | SEQ ID NO: 68 |
| c19-3-10.seq | + | | SEQ ID NO: 69 |
| c19-4-19.seq | + | | SEQ ID NO: 70 |
| c19-4-22.seq | + | | SEQ ID NO: 71 |
| c19-5-1.seq | − | | SEQ ID NO: 72 |
| c19-5-4.seq | − | | SEQ ID NO: 73 |
| c19-6-3.seq | + | | SEQ ID NO: 74 |
| c19-7-4.seq | + | | putative translation initiation factor 2B beta subunit (NIFb) EIF2B beta homolog Nicotiana tabacum |
| | | | (gb\|AF137288) SEQ ID NO: 75 |
| c2-1-10.seq | − | | SEQ ID NO: 76 |
| c2-11-14.seq | + | | SEQ ID NO: 77 |
| c2-11-2.seq | + | | SEQ ID NO: 78 |
| c2-2-1.seq | + | | SEQ ID NO: 79 |
| c2-2-3.seq | + | | SEQ ID NO: 80 |
| c2-4-1.seq | + | | SEQ ID NO: 81 |
| c2-5-6.seq | + | | SEQ ID NO: 82 |
| c2-6-5.seq | − | | SEQ ID NO: 83 |
| c2-7-1.seq | + | | nonsucrose-inducible patatin precursor-strand Solanum brevidens (gb\|U09331) SEQ ID NO: 84 |
| c2-9-14.seq | − | | SEQ ID NO: 85 |
| c20-1-4.seq | + | | DNA-binding protein (pabf) Nicotiana tabacum (gb\|U06712) SEQ ID NO: 86 |
| c3-2-4.seq | + | | SEQ ID NO: 87 |
| c3-3-6.seq | + | | SEQ ID NO: 88 |
| c3-4-1.seq | − | | SEQ ID NO: 89 |
| c4-1-2.seq | + | | SEQ ID NO: 90 |
| c4-3-3.seq | + | | SEQ ID NO: 91 |
| c5-1-2.seq | + | | SEQ ID NO: 92 |
| c6-8-13.seq | + | | SEQ ID NO: 93 |
| c6-8-4.seq | + | | SEQ ID NO: 94 |
| c6-8-9.seq | + | | SEQ ID NO: 95 |
| c7-1-2.seq | − | | SEQ ID NO: 96 |
| c7-1-6.seq | − | | SEQ ID NO: 97 |
| c7-3-10.seq | − | | SEQ ID NO: 98 |
| c7-3-3.seq | − | | hypothetical protein Arabidopsis thaliana (emb\|CAB62623) SEQ ID NO: 99 |
| c7-3-9.seq | − | | SEQ ID NO: 100 |
| c8-1-5.seq | + | | SEQ ID NO: 101 |
| c9-1-4.seq | + | | hypothetical protein Arabidopsis thaliana (dbj\|BAB08809) SEQ ID NO: 102 |
| g10-1-1.seq | + | | putative ABA-responsive protein Arabidopsis thaliana (dbj\|BAB11190) SEQ ID NO: 103 |
| g12-1-21.seq | − | | hypothetical protein Arabidopsis thaliana (pir\|T01731) SEQ ID NO: 104 |
| g12-1-5.seq | − | | Putative membrane-related protein Arabidopsis thaliana (gb\|AAD38248) SEQ ID NO: 105 |
| g14-2-4.seq | + | + | vetispiradiene synthase Solanum tuberosum (gb\|AAD02223) SEQ ID NO: 106 |
| g14-3-10.seq | + | | SEQ ID NO: 107 |
| g14-3-22.seq | + | | hypothetical protein Spinacia oleracea (pir\|T09217) SEQ ID NO: 108 |
| g14-3-3.seq | + | | Sequence 162 from Patent EP0953640 Nicotiana tabacum (emb\|AX014606) SEQ ID NO: 109 |
| g14-3-4.seq | + | | HR associated Ca2+-binding protein Phaseolus vulgaris (gb\|AAD47213) SEQ ID NO: 110 |
| g14-3-7.seq | + | | SEQ ID NO: 111 |
| g15-1-37.seq | + | | putative golgi transport complex protein Arabidopsis thaliana (gb\|AAF16568) SEQ ID NO: 112 |
| g15-2-2.seq | + | = | ubiquitin Nicotiana tabacum (gb\|U66264) able to induce HR-like lesions SEQ ID NO: 113 |
| g15-3-11.seq | − | | Sequence 7 from Patent EP0953640 Nicotiana tabacum (emb\|AX014451) SEQ ID NO: 114 |
| g15-3-7.seq | − | | SEQ ID NO: 115 |
| g15-4-1.seq | + | | SEQ ID NO: 116 |
| g17-2-13.seq | + | + | WRKY DNA binding protein Solanum tuberosum (emb\|CAB97004) SEQ ID NO: 117 |
| g17-3-2.seq | + | | SEQ ID NO: 118 |
| g18-4-7.seq | + | | putative ribosomal protein L18 (60S) Arabidopsis thaliana (gb\|AAF26138) SEQ ID NO: 119 |
| g18-5-1.seq | − | | SEQ ID NO: 120 |
| g18-5-12.seq | − | | SEQ ID NO: 121 |
| g18-6-12.seq | + | | SEQ ID NO: 122 |
| g18-6-5.seq | + | | SEQ ID NO: 123 |
| g18-7-5.seq | + | | SEQ ID NO: 124 |
| g18-8-7.seq | + | | SEQ ID NO: 125 |
| g19-1-5.seq | − | | unknown protein Arabidopsis thaliana (gb\|AAF23197) SEQ ID NO: 126 |
| g19-1-6.seq | + | | SEQ ID NO: 127 |
| g19-1-7.seq | + | | putative protein Arabidopsis thaliana (emb\|CAB82697) SEQ ID NO: 128 |
| g19-2-1.seq | + | | SEQ ID NO: 129 |
| g19-2-9.seq | + | | SEQ ID NO: 130 |
| g2-1-2.seq | + | + | 5-epi-aristolochene synthase Nicotiana tabacum (emb\|Y08847) SEQ ID NO: 131 |
| g20-2-20.seq | + | | hypothetical protein Arabidopsis thaliana (gb\|AAF14679) SEQ ID NO: 132 |
| g20-2-29.seq | + | | SEQ ID NO: 133 |
| g20-2-31.seq | + | | SEQ ID NO: 134 |
| g3-1-1.seq | + | | ankyrin-like protein Arabidopsis thaliana (dbj\|BAB10271) SEQ ID NO: 135 |
| g3-1-4.seq | + | = | ADP-ribosylation factor Capsicum annuum (gb\|AAF65512) SEQ ID NO: 136 |
| g6-2-13.seq | + | + | leucoanthocyanidin dioxygenase 2, putative; 51024-52213 Arabidopsis thaliana (gb\|AAG21532) |
| | | | SEQ ID NO: 137 |
| g6-3-7.seq | + | + | ATP citrate lyase Arabidopsis thaliana (dbj\|BAB09916) SEQ ID NO: 138 |
| g6-4-4.seq | + | | SEQ ID NO: 139 |
| g6-4-5.seq | + | | ATP-dependent protease proteolytic subunit ClpP-like protein Arabidopsis thaliana (dbj\|BAB09831) |
| | | | SEQ ID NO: 140 |
| g7-1-1.seq | + | | RNA-binding protein MEI2 (meiotic regulator), putative; 36123-32976 Arabidopsis thaliana |
| | | | (gb\|AAG12640) SEQ ID NO: 141 |
| g7-1-4.seq | + | | SEQ ID NO: 142 |
| g9-2-2.seq | + | + | P-glycoprotein-like protein Arabidopsis thaliana (emb\|CAB71875) SEQ ID NO: 143 |
| g9-2-6.seq | + | | SEQ ID NO: 144 |
| g9-3-17.seq | + | | SEQ ID NO: 145 |
| g9-3-4.seq | + | | SEQ ID NO: 146 |
| g9-5-5.seq | + | | SEQ ID NO: 147 |
| g9-6-1.seq | + | + | lipoxygenase Solanum tuberosum (gb\|AAD09202) SEQ ID NO: 148 |
| t12-1-7.seq | + | + | serine/threonine/tyrosine-specific protein kinase APK1A Arabidopsis thaliana (sp\|Q06548) SEQ ID |
| | | | NO: 149 |
| t12-2-1.seq | + | | chitinase class 4 Vigna unguiculata (pir\|S57476) SEQ ID NO: 150 |
| t12-2-18.seq | + | | SEQ ID NO: 151 |
| t18-2-5.seq | + | + | basic PRB-1b Nicotiana tabacum (emb\|X66942) SEQ ID NO: 152 |
| t18-3-2.seq | + | | SEQ ID NO: 153 |
| t18-3-6.seq | + | | RNA-or ssDNA-binding protein Vicia faba (pir\|T12196) SEQ ID NO: 154 |
| t18-4-18.seq | − | | ADP-glucose pyrophosphorylase small subunit Solanum tuberosum (emb\|X55650) SEQ ID NO: 155 |
| t-2-1-1.seq | + | | ubiquitin carrier protein Lycopersicon esculentum (sp\|P35135) SEQ ID NO: 156 |
| t2-1-3.seq | + | | Hypothetical protein chlP Nicotiana tobacum (sp\|P12204) SEQ ID NO: 157 |
| t2-6-3.seq | + | | SEQ ID NO: 158 |
| t7-1-12.seq | + | = | Hypothetical protein Arabidopsis thaliana (gb\|AAF26468) SEQ ID NO: 159 |
| t7-1-14.seq | + | | t7-2-4.seq + intron SEQ ID NO: 167 |
| t7-2-4.seq | + | + | Multifunctional protein of glyoxysomal fatty acid beta-oxidation Brassica napus (emb\|AJ000886) |
| | | | SEQ ID NO: 161 |
| t7-4-7.seq | + | | putative glutathione S-transferase; 80986-80207 Arabidopsis thaliana (gb\|AAF15930) SEQ ID |
| | | | NO: 161 |
| t7-4-8.seq | + | | SEQ ID NO: 162 |
| t7-5-4.seq | + | | SEQ ID NO: 163 |
| t7-5-5.seq | + | | SEQ ID NO: 164 |
| t7-6-4.seq | + | | SEQ ID NO: 165 |

DD+ = induced on differential display gel
DD− = repressed on differential display gel
N+ = induced on Northern
N− = repressed on Northern
N= = constant on Northern

All GenBank, or other databases, references cited herein are hereby incorporated by reference to the same extent as if each reference were individually and specifically indicated to be incorporated by reference and were set forth in its entirety herein.

TABLE 2
Genes isolated by differential display with induction confirmed by Northern Analysis.
Columns refer, respectively, to the clone number, the name of the predicted gene,
the length of isolated cDNA including both primers, the length of deduced partial
protein sequence, the (putative) homologue with highest e-value identified in the
database, accession number of a (putative) homologue, percentage of the amino
acid sequence identity (superscript indicates homology of the same segment to
similar domains localized upstream⁽¹⁾ and downstream⁽²⁾ in the
homologous protein), and the length of the high-scoring segment pair(s)
identified by blastx homology search.

| | | cDNA | Peptide | | | | HSPS |
|---|---|---|---|---|---|---|---|
| Clone | cDNA/gene | length | length | | Accession | % sequence | length |
| Number | name | (bp) | (aa) | (Putative) homologue | Number | identity (aa) | (aa) |
| T18-2-5 | PRB-1b | 448 | 48 | pathogenesis-related protein 1b, PRB-1b | emb\|X66942 | 100% | 47 |
| | | | | Nicotiana tabacum) SEQ ID NO: 152 | | | |
| C15-1-4 | CBP20 | 508 | 84 | pathogen- and wound-inducible antifungal | gb\|AAB29959 | 98% | 84 |
| | | | | protein CBP20 (clone cbp20-52) (Nicotiana | | | |
| | | | | tabacum) SEQ ID NO: 52 | | | |
| G2-1-2 | EAS | 228 | 8 | 5-epi-aristolochene synthase (clone str319) | emb\|Y08847 | 100% | 7 |
| | | | | (Nicotiana tabacum) SEQ ID NO: 131 | | | |
| G14-2-4 | VS | 382 | 66 | vetispiradiene synthase (Solanum tuberosum) | gb\|AAD02223 | 100% | 65 |
| | | | | SEQ ID NO: 106 | | | |
| G6-3-7 | ATPC-L | 397 | 49 | ATP citrate-lyase (Arabidopsis thaliana) | dbj\|BAB09916 | 97% | 48 |
| | | | | SEQ ID NO: 138 | | | |
| C18-1-2 | DNAJ | 397 | 89 | DnaJ-like protein (Arabidopsis thaliana) | emb\|CAB86070 | 75% | 88 |
| | | | | SEQ ID NO: 66 | | | |
| G9-2-2 | MDR | 505 | 96 | P-glycoprotein-like protein (Arabidopsis | emb\|CAB71875 | 68%⁽¹⁾ | 91 |
| | | | | thaliana), nucleotide binding fold (NBF2 | | 91%⁽²⁾ | 95 |
| | | | | SEQ ID NO: 143 | | | |
| G6-2-13 | DIOX | 525 | 96 | Leucoanthocyanidin dioxygenase 2-like | gb\|AAG21532 | 80% | 92 |
| | | | | protein (Arabidopsis thaliana) SEQ ID | | | |
| | | | | NO: 137 | | | |
| G9-6-1 | Lox2 | 269 | 19 | Lipoxygenase (Solanum tuberosum) SEQ ID | gb\|AAD09202 | 100% | 17 |
| | | | | NO: 148 | | | |
| T7-2-4 | MFP | 413 | 55 | multifunctional protein of glyoxysomal fatty | emb\|AJ000886 | 61% | 46 |
| | | | | acid beta-oxidation (Brassica napus) SEQ ID | | | |
| | | | | NO: 160 | | | |
| T12-1-7 | TPK | 361 | 75 | protein tyrosine-serine-threonine kinase | sp\|Q06548 | 36% | 82 |
| | | | | APK1A (Arabidopsis thaliana) SEQ ID | | | |
| | | | | NO: 149 | | | |
| G17-2-13 | WRKY11 | 548 | 87 | WRKY DNA binding protein (Solanum | emb\|CAB97004 | 94% | 86 |
| | | | | tuberosum) SEQ ID NO: 117; SEQ ID | | | |
| | | | | NO: 168; and SEQ ID NO: 169 | | | |

All GenBank, or other databases, references cited herein are hereby incorporated by reference to the same extent as if each reference were individually and specifically indicated to be incorporated by reference and were set forth in its entirety herein.

### EXAMPLE IV

### MV-Induced Genes are Regulated Differently during the Treatment

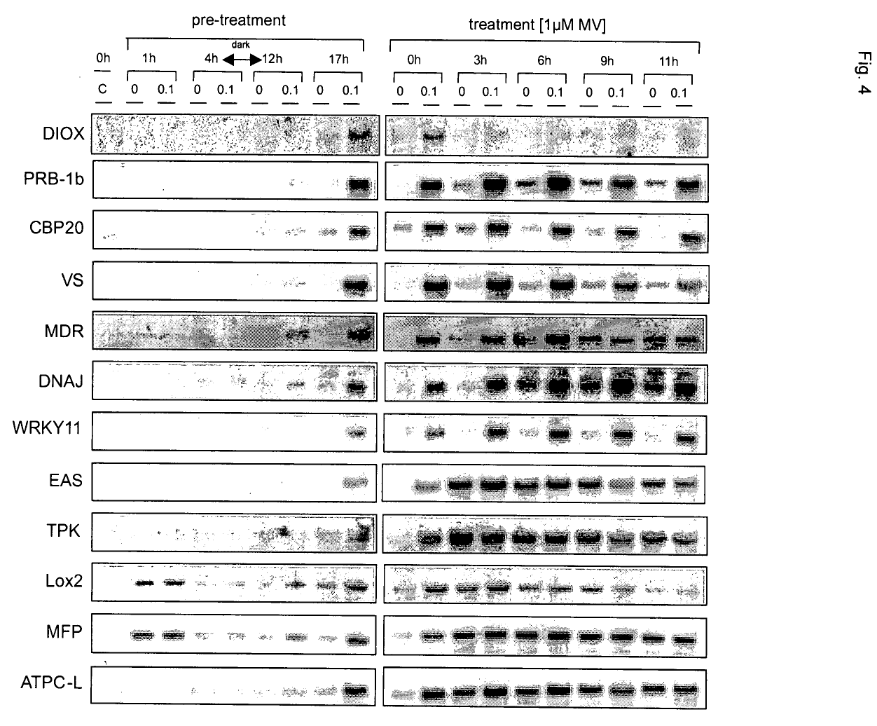

Of the antioxidant genes tested, only expression of Gpx and SodCc correlated with enhanced tolerance of pretreated samples (FIG. 3). To further investigate the transcriptional response of genes induced during adaptation to MV, Northern hybridizations were performed for a subset of identified genes (Table 2) during the pretreatment and the treatment (FIG. 4). The earliest gene induction could be observed already after one hour of the pretreatment for MFP and Lox2 and is likely related to the wounding of the tissue during the leaf disc preparation. Lipoxygenase (Lox) and multifunctional protein (MFP) are both implicated in a pathway leading to lipid breakdown products such as jasmonic acid, and wounding may induce their expression (Mueller, 1997). This induction was transient and was seen in both water reference samples and MV-pretreated samples.

During the first four hours of the pretreatment, there was no discernible induction of gene expression by MV, while during the treatment, the induction was already visible after three hours. The concentration of MV during the treatment was ten times higher, suggesting that the timing of induction is concentration-dependent. All genes, except DIOX, were induced after 12 hours of the pretreatment with 0.1 μM MV, but more detailed time course analysis would be required to determine exact timing of induction. The low level of induction at this time point probably reflects the preceding dark period of eight hours with no photosynthetic activity. The primary site of action of MV in photosynthesizing plants are the chloroplasts (Halliwell and Gutteridge, 1989) and active photosynthesis is required for maximal generation of superoxide by this redox-cycling compound. This is in agreement with the further and much stronger induction of the mRNA level on the light during the last five hours of the pretreatment.

Expression of all genes, except DIOX, was further induced during the treatment with 1 μM MV and the induction started within the first three hours of the treatment. In the course of the treatment, two different expression patterns were essentially recognized.

For one group of genes (PRB-1b, CBP20, VS, MDR, DNAJ and WRKY11), expression was induced by a 1 μM MV treatment in both the 0.1 μM MV-pretreated samples and water reference samples such that the level of transcript remained higher in the 0.1 μM MV-pretreated samples for at least six hours, which is the time when the difference in tolerance between pretreated and nonpretreated samples began to be manifested. The increase in transcript levels with time was rather slow, reaching the maximum between six and nine hours in water reference samples, while it was generally three hours earlier in MV-pretreated samples. Towards the end of the treatment, the transcript level declined. A similar expression pattern was observed for antioxidant genes GPx and SodCc (FIG. 3).

The second group of genes (EAS, TPK, Lox2 and MFP) was also transcriptionally induced by a 1 μM MV treatment (except Lox2 in MV-pretreated samples) but with different kinetics. The induction was much stronger in the water reference samples, so the differences in mRNA level between MV-pretreated and the water reference samples diminished. The response was also faster, with transcript levels reaching a maximum within three hours (six hours for MFP) in both water reference and MV-pretreated samples. The kinetics of ATPC-L expression had rather intermediate character with respect to the expression patterns of the two described gene groups. Together, these data indicate the presence of at least two different mechanisms for activation of defense genes by MV.

### EXAMPLE V

### Overexpression of WRKY11 Provokes Oxidative Stress Tolerance

Full-length cDNA sequence was obtained by 5′ RACE using total leaf RNA and a gene-specific 3′ primer.

The corresponding gene was designated WRKY11 (SEQ ID NOS:168 and 169) because ten nonidentical tobacco WRKY genes were already present in the database.

WRKY proteins are divided into three classes based on type and number of WRKY domains. WRKY family members show only little homology among each other outside of the WRKY domains (Eulgem, Rushton et al., 2000). Database search (blastx on nrprot) revealed only one protein that is significantly similar to WRKY1 (SEQ ID NO: 169) within the N-terminal part of the protein: StWRKIY1 from potato (Dellagi, Heilbronn et al., 2000).

Segregating populations (F2) of A. thaliana plants (C 24) transformed with WRKY11 under control of the 35S promoter (35S—WRKY11) or with WRKY11 fused to the VP16 activation domain under control of the 35S promoter (35S—WRKY11-VP16) were grown on MS media with kanamycine. Approximately three-week-old seedlings resistant to kanamycine from 3:1 segregating lines (WV4 and WV9 WRKY11-VP16 transformed lines) were transferred to the solid media containing ½ MS salts, 1% sucrose and 2 μM methyl viologen (MV) or on plates without MV. As control plants, untransformed A. thaliana plants were used (C24). After three to four weeks, phenotypic differences were assessed.

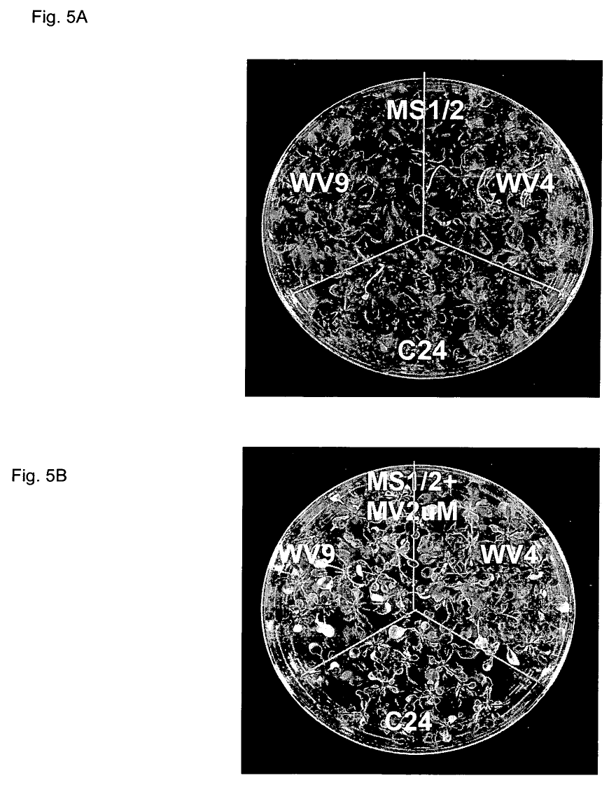

On control plates without MV, no difference in growth between WRKY11-VP16 transformants and controls were observed (FIG. 5A). On plates containing MV, growth of all plants was retarded; however, differences in growth and MV tolerance between lines overexpressing WRKY11 and control plants were observed.

Line WV4 was more tolerant to MV than untransformed Arabidopsis control (C24). However, line WV9 did not differ significantly from control in its growth and MV tolerance (FIG. 5B).

### REFERENCES

Altschul, S. F., Madden, T. L., Schaffer, A. A., Zhang, J., Zhang, Z., Miller, W., and Lipman, D. J. (1997) Gapped BLAST and PSI-BLAST: a new generation of protein database search programs. Nucleic Acids Res. 25, 3389-3402.

Alvarez, M. E., Pennell, R. I., Meijer, P-J., Ishikawa, A., Dixon, R. A. and Lamb, Ch. (1998) Reactive oxygen intermediates mediate a systemic signal network in the establishment of plant immunity. Cell 92, 773-784.

Banzet, N., Richaud, Ch., Deveaux, Y., Kazmaier, M., Gagnon, J. and Triantaphylides, Ch. (1998) Accumulation of small heat shock proteins, including mitochondrial HSP22, induced by oxidative stress and adaptive reponses in tomato cells. Plant Journal 13, 519-527.

Bartels, D., Furini, A., Ingram, J. and Salamini, F. (1996) Responses of plants to dehydration stress: a molecular analysis. Plant Growth Regulation 20, 111-118.

Bowler, C., Slooten, L., Vandenbranden, S., De Rycke, R., Botterman, J., Sybesma, C., Van Montagu, M. and Inze, D. (1991) Manganese superoxide dismutase can reduce cellular damage mediated by oxygen radicals in transgenic plants. EMBO J. 10,1723-1732.

Chamnongpol, S., Willekens, H., Moeder, W., Langebartels, Ch., Sandermann, H., Van Montagu, M., Inze, D. and Van Camp, W. (1998) Defense activation and enhanced pathogen tolerance induced by H₂O₂ in transgenic tobacco. Proc. Natl. Acad. Sci. 95, 5818-5823.

Clough, S. J. and Bent, A. F. (1998) Floral dip: a simplified method for Agrobacterium-mediated transformation of Arabidopsis thaliana. Plant J. 16, 735-743.

Cloutier, Y. and Andrews Ch. J. (1984) Efficiency of Cold Hardiness Induction by Dessication Stress in Four Winter Cereals. Plant Physiol. 76, 595-598.

Criqui, M. C., Jamet, E., Parmentier, Y., Marbach, J., Durr, A. and Fleck, J. (1992) Isolation and characterization of a plant cDNA showing homology to animal glutathione peroxidases. Plant. Mol. Biol. 18, 623-627.

Davies, J. M. S., Lowry, C. V. and Davies, K. J. A. (1995) Transient adaptation to oxidative stress in yeast. Arch. Biochem. Biophys. 327, 1-6.

Dellagi, A., J. Heilbronn, et al. (2000). A potato gene encoding a WRKY-like transcription factor is induced in interactions with Erwinia carotovora subsp atroseptica and Phytophthora infestans and is coregulated with class I endochitinase expression. Molecular Plant-Microbe Interactions 13, 1092-1101.

Demple, B. (1991) Regulation of bacterial oxidative stress genes. Annu. Rev. Genet. 25, 315-337.

Eulgem, T., P. J. Rushton, et al. (2000). The WRKY superfamily of plant transcription factors. Trends in Plant Science 5, 199-206.

Godon, Ch., Lagniel, G., Lee, J., Buhler, J-M., Kieffer, S., Perrot, M., Boucherie, H., Toledano, M. B. and Labarre, J. (1998). The H₂O₂Stimulon in Saccharomyces cerevisiae. 273, 22480-22489.

Halliwell B., and Gutteridge, J. M. C. (1989). Free Radicals in Biology and Medicine. Oxford: Calrendon Press.

Henikoff, S. and Henikoff, J. G. (1992). Amino acid substitution matrices from protein blocks. Proc. Natl. Acad. Sci. 89, 10915-10919.

Inzé, D. and Van Montagu, M. (1995). Oxidative stress in plants. Current Opinion in Biotechnology 6, 153-158.

Jansen, M. A. K., Gaba, V. and Greenberg, B. M. (1998). Higher plants and UV-B radiation: balancing damage, repair and acclimation. Trends in Plant Science 3, 131-135.

Jamieson, D. J. (1992) Saccharomyces cerevisiae has distinct adaptive responses to both hydrogen peroxide and menadione. Journal of Bacteriology 174, 6678-6681.

Karpinski, S., Reynolds, H., Karpinska, B., Wingsle, G., Creissen, G., Mullineaux, P. (1999). Systemic signaling and acclimation in response to excess excitation energy in Arabidopsis. Science 284, 654-657.

Keller, E. and Steffen, K. L. (1995). Increased chilling tolerance and altered carbon metabolism in tomato leaves following application of mechanical stress. Physiologia Plantarum 93, 519-525.

Levine, A., Tenhaken, R., Dixon, R. and Lamb, C. (1994). H₂O₂ from oxidative burst orchestrates the plant hypersensitive disease resistance response. Cell 79, 583-593.

Maniatis. T., Fritsch, E. F. and Sambrook, J. (1982) Molecular Cloning, A Laboratory Manual. Cold Spring Harbor Laboratory, Cold Spring Harbor, N.Y.

Mueller, M. J. (1997). Enzymes involved in jasmonic acid biosynthesis. Physiol. Plant. 100, 653-663.

Örvar, B. J., McPherson, J. and Ellis, B. E. (1997). Pre-activating wounding response in tobacco prior to high-level ozone exposure prevents necrotic injury. The Plant Journal 11, 1997.

Orzech, K. A. and Burke, J. J. (1988). Heat shock and the protection against metal toxicity in wheat leaves. Plant, Cell and Environment 11, 711-714.

Prasad, T. K., Anderson, M. D., Martin, B. A., Steward, C. R. (1994). Evidence for chilling-induced oxidative stress in maize seedlings and a regulatory role for hydrogen peroxide. The Plant Cell 6, 65-74.

Sandermann, H., Jr., Ernst, D., Heller, W. and Langebartels, Ch. (1998). Ozone: an abiotic elicitor of plant defence reactions. Trends in Plant Science, 3, 47-50.

Seppänen, M. M., Majaharju, M. Somersalo, S. and Pehu, E. (1998) Freezing tolerance, cold acclimation and oxidative stress in potato. Paraquat tolerance is related to acclimation but is a poor indicator of freezing tolerance. Physiologia Plantarum 102, 454-460.

Shaul, O., Mironov, V., Burssens, S., Van Montagu, M. and Inze, D. (1996) Two Arabidopsis cyclin promoters mediate distinctive transcriptional oscillation in synchronized tobacco BY-2 cells. Proc. Natl. Acad. Sci. 93, 4868-4872.

Somsschich, I. E. and Hahlbrock, K. (1998). Pathogen defence in plants—a paradigm of biological complexity. Trends in Plant Science 3, 86-90.

Sticher, L., Mauch-Mani, B. and Métraux, J. P. (1997). Systemic acquired resistance. Annu. Rev. Phytopatol. 35, 235-270.

Strobel, N. E. and Kuc, J. A. (1995). Chemical and biological inducers of systemic resistance to pathogens protect cucumber and tobacco plants from damage caused by paraquat and cupric chloride. Phytopathology 85, 1306-1310.

Storz, G., Tartaglia, L. A., Farr, S. B. and Ames, B. N. (1990). Bacterial defenses against oxidative stress. Trends in Genetics 6, 363-368.

Tsang, E. W. T., Bowler, Ch., Hérouart, D., Van Camp, W., Villarroel, R., Genetello, Ch., Van Montagu, M. and Inze, D. (1991). Differential regulation of superoxide dismutases in plants exposed to environmental stress. The Plant Cell 3, 783-792.

Vierling, E. (1991). The roles of heat shock proteins in plants. Annu. Rev. Plant Physiology Plant Mol. Biol. 42, 579-620.

Willekens, H., Villarroel, R., Van Montagu, M., Inze, D. and Van Camp, W. (1994). Molecular identification of catalases from Nicotiana plumbaginifolia (L.) FEBS Lett. 352, 79-83.

What is claimed is:

1. A method of isolating a stress-regulated nucleic acid sequence of interest, said method comprising:
isolating plant material;
inducing stress adaptation in said isolated plant material by application of a sublethal stress;
identifying differential expression of a sequence between stress-adapted and nonadapted plant material; and
isolating a differentially expressed sequence of interest.

2. The method according to claim 1, wherein said induction of stress adaptation is produced by a methyl viologen pretreatment or treatment.

3. The method according to claim 1, wherein said isolated plant material is tobacco leaf material.

4. The method according to claim 1, wherein isolating said differentially expressed sequence is at least partly performed by amplifying said differentially expressed sequence by PCR.

5. The method according to claim 1, wherein said differentially expressed sequence of interest is a nucleic acid sequence.

6. An isolated nucleic acid comprising a nucleic acid encoding the polypeptide sequence set forth in SEQ ID NO: 169.

7. A sequence of interest, produced by a process comprising:
isolating plant material;
inducing stress adaptation in said isolated plant material by application of a sublethal stress;
identifying differential expression of a sequence between stress-adapted and nonadapted plant material; and
isolating said differentially expressed sequence.

8. The sequence of interest of claim 7, wherein said differentially expressed sequence is selected from the group of sequences consisting of SEQ ID NO: 1 to SEQ ID NO: 167.

9 The sequence of interest of claim 7, wherein said differentially expressed sequence encodes a protein comprising SEQ ID NO: 169.

10. The sequence of interest of claim 7, wherein said differentially expressed sequence comprises SEQ ID NO:168.

11. A method of modulating plant stress tolerance, said method comprising;
isolating plant material;
inducing stress adaptation in said isolated plant material by application of a sublethal stress;
identifying differential expression of a sequence between stress-adapted and nonadapted plant material;
isolating a: differentially expressed sequence, or a sequence of interest that is at least 60% identical to said differentially expressed sequence,
introducing said differentially expressed sequence into a vector;
introducing said vector into a plant cell; and
expressing said differentially expressed sequence, thereby modulating plant stress tolerance.

12. The method according to claim 11, wherein said differentially expressed sequence is selected from the group of sequences consisting of SEQ ID NO: 1 to SEQ ID NO: 167.

13. The method according to claim 11, wherein said differentially expressed sequence encodes a protein comprising SEQ ID NO: 169.

14. The method according to claim 11, wherein said differentially expressed sequence comprises SEQ ID NO: 168.

15. The method according to claim 11, further comprising:
identifying a genomic DNA sequence corresponding to a 5′ end of said differentially expressed sequence; and
identifying a promoter sequence in said genomic DNA.

16. The method according to claim 0.12, further comprising:
identifying a full length cDNA sequence for said differentially expressed sequence.

17. The method according to claim 11, wherein said sequence of interest is 90% identical to said differentially expressed sequence.

18. The sequence of interest of claim 7, further comprising:
identifying genomic DNA corresponding to a 5′ end of said differentially expressed sequence; and
identifying a promoter sequence in said genomic DNA.

19. The method according to claim 11, wherein stress adaptation is induced by a methyl viologen pretreatment or treatment.

20. The method according to claim 11, wherein said isolated plant material is tobacco.

21. The sequence of interest of claim 7, further comprising inserting said differentially expressed sequence of interest into a vector.

22. The method according to claim 11, further comprising introducing said differentially expressed sequence of interest into a vector and introducing said vector into a plant cell, thereby producing a plant cell having increased stress tolerance.

23. A plant comprising the plant cell of claim 22.
